SEQUENCE LISTING (23 sequences)

SEQ ID NO: 1 - protein, 167 aa, Homo sapiens
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 2 - protein, 117 aa, Homo sapiens
MPSPGTVCSLLLLGMLWLDLAMAGSSFLSPEHQRVQQRKESKKPPAKLQPRALAGWLRPEDGGQAEGAEDELEVRFNAPFDVGIKLSGVQYQQHSQALGKFLQDILWEEAKEAPADK

SEQ ID NO: 3 - protein, 366 aa, Homo sapiens
MWNATPSEEPGFNLTLADLDWDASPGNDSLGDELLQLFPAPLLAGVTATCVALFVVGIAGNLLTMLVVSRFRELRTTTNLYLSSMAFSDLLIFLCMPLDLVRLWQYRPWNFGDLLCKLFQFVSESCTYATVLTITALSVERYFAICFPLRAKVVVTKGRVKLVIFVIWAVAFCSAGPIFVLVGVEHENGTDPWDTNECRPTEFAVRSGLLTVMVWVSSIFFFLPVFCLTVLYSLIGRKLWRRRRGDAVVGASLRDQNHKQTVKMLAVVVFAFILCWLPFHVGRYLFSKSFEPGSLEIAQISQYCNLVSFVLFYLSAAINPILYNIMSKKYRVAVFRLLGFEPFSQRKLSTLKDESSRAWTESSINT

SEQ ID NO: 4 - protein, 180 aa, Homo sapiens
MKSIYFVAGLFVMLVQGSWQRSLQDTEEKSRSFSASQADPLSDPDQMNEDKRHSQGTFTSDYSKYLDSRRAQDFVQWLMNTKRNRNNIAKRHDEFERHAEGTFTSDVSSYLEGQAAKEFIAWLVKGRGRRDFPEEVAIVEELGRRHADGSFSDEMNTILDNLAARDFINWLIQTKITDRK

SEQ ID NO: 5 - protein, 477 aa, Homo sapiens
MPPCQPQRPLLLLLLLLACQPQVPSAQVMDFLFEKWKLYGDQCHHNLSLLPPPTELVCNRTFDKYSCWPDTPANTTANISCPWYLPWHHKVQHRFVFKRCGPDGQWVRGPRGQPWRDASQCQMDGEEIEVQKEVAKMYSSFQVMYTVGYSLSLGALLLALAILGGLSKLHCTRNAIHANLFASFVLKASSVLVIDGLLRTRYSQKIGDDLSVSTWLSDGAVAGCRVAAVFMQYGIVANYCWLLVEGLYLHNLLGLATLPERSFFSLYLGIGWGAPMLFVVPWAVVKCLFENVQCWTSNDNMGFWWILRFPVFLAILINFFIFVRIVQLLVAKLRARQMHHTDYKFRLAKSTLTLIPLLGVHEVVFAFVTDEHAQGTLRSAKLFFDLFLSSFQGLLVAVLYCFLNKEVQSELRRRWHRWRLGKVLWEERNTSNHRASSSPGHGPPSKELQFGRGGGSQDSSAETPLAGGLPRLAESPF

SEQ ID NO: 6 - protein, 1071 aa, Homo sapiens
AGCGGCGGCGAAGGAGGGGAAGAAGAGCCGCGACCGAGAGAGGCCGCCGAGCGTCCCCGCCCTCAGAGAGCAGCCTCCCGAGACAGAGCCTCAGCCTGCCTGGAAGATGCCGAGATCGTGCTGCAGCCGCTCGGGGGCCCTGTTGCTGGCCTTGCTGCTTCAGGCCTCCATGGAAGTGCGTGGCTGGTGCCTGGAGAGCAGCCAGTGTCAGGACCTCACCACGGAAAGCAACCTGCTGGAGTGCATCCGGGCCTGCAAGCCCGACCTCTCGGCCGAGACTCCCATGTTCCCGGGAAATGGCGACGAGCAGCCTCTGACCGAGAACCCCCGGAAGTACGTCATGGGCCACTTCCGCTGGGACCGATTCGGCCGCCGCAACAGCAGCAGCAGCGGCAGCAGCGGCGCAGGGCAGAAGCGCGAGGACGTCTCAGCGGGCGAAGACTGCGGCCCGCTGCCTGAGGGCGGCCCCGAGCCCCGCAGCGATGGTGCCAAGCCGGGCCCGCGCGAGGGCAAGCGCTCCTACTCCATGGAGCACTTCCGCTGGGGCAAGCCGGTGGGCAAGAAGCGGCGCCCAGTGAAGGTGTACCCTAACGGCGCCGAGGACGAGTCGGCCGAGGCCTTCCCCCTGGAGTTCAAGAGGGAGCTGACTGGCCAGCGACTCCGGGAGGGAGATGGCCCCGACGGCCCTGCCGATGACGGCGCAGGGGCCCAGGCCGACCTGGAGCACAGCCTGCTGGTGGCGGCCGAGAAGAAGGACGAGGGCCCCTACAGGATGGAGCACTTCCGCTGGGGCAGCCCGCCCAAGGACAAGCGCTACGGCGGTTTCATGACCTCCGAGAAGAGCCAGACGCCCCTGGTGACGCTGTTCAAAAACGCCATCATCAAGAACGCCTACAAGAAGGGCGAGTGAGGGCACAGCGGGCCCCAGGGCTACCCTCCCCCAGGAGGTCGACCCCAAAGCCCCTTGCTCTCCCCTGCCCTGCTGCCGCCTCCCAGCCTGGGGGGTCGTGGCAGATAATCAGCCTCTTAAAGCTGCCTGTAGTTAGGAAATAAAACCTTTCAAATTTCACA

SEQ ID NO: 7 - protein, 97 aa, Homo sapiens
MVFVRRPWPALTTVLLALLVCLGALVDAYPIKPEAPGEDASPEELNRYYASLRHYLNLVTRQRYGKRDGPDRLLSKTFFPDGEDRPVRSRSEGPDLW

SEQ ID NO: 8 - protein, 402 aa, Homo sapiens [sequence omitted]

SEQ ID NO: 9 - protein, 131 aa, Sus scrofa
MDVTRLLLATLLVCLCFFTASSHLAPEEKSKDERSLRSNSSMNLLDFPSVSIVALNKKSKKISRKEAEKRSSKKKASMKKVAQPRPPRPAPCVANRDSCKPPALACCDPCAFCQCRFFRSACSCRVLNPTC

SEQ ID NO: 10 - protein, 132 aa, Homo sapiens
MLTAAVLSCALLLALPATRGAQMGLAPMEGIRRPDQALLPELPGLGLRAPLKKTTAEQAEEDLLQEAQALAEVLDLQDREPRSSRRCVRLHESCLGQQVPCCDPCATCYCRFFNAFCYCRKLGTAMNPCSRT

SEQ ID NO: 11 - protein, 317 aa, Pan troglodytes
MAVQGSQRRLLGSLNSTPTAIPQLGLAANQTGARCLEVSIPDGLFLSLGLVSLVENMLVVATIAKNRNLHSPMYCFICCLALSDLLVSGSNVLETAVILLLEAGALVARAAVLQQVDNVIDVITCSSMLSSLCFLGAIAVDRYISIFYALRYHSIVTLPRARRAIAAIWVASVLFSTLFIAYCDHTAVLLCLVVFFLAVLVLMAVLYVHMLARACQHAQGIARLHKRQRPVHQGFGLKGAVTLTILLGIFFLCWGPFFLHLTLIVLCPEHPTCGCIFKNFNLFLALIICNAIIDPLIYAFHSQELRRTLKEVLTCSW

SEQ ID NO: 12 - protein, 297 aa, Homo sapiens
MKHIINSYENINNTARNNSDCPRVVLPEEIFFTISIVGVLENLIVLLAVFKNKNLQAPMYFFICSLAISDMLGSLYKILENILIILRNMGYLKPRGSFETTADDIIDSLFVLSLLGSIFSLSVIAADRYITIFHALRYHSIVTMRRTVVVLTVIWTFCTGTGITMVIFSHHVPTVITFTSLFPLMLVFILCLYVHMFLLARSHTRKISTLPRANMKGAITLTILLGVFIFCWAPFVLHVLLMTFCPSNPYCACYMSLFQVNGMLIMCNAVIDPFIYAFRSPELRDAFKKMIFCSRYW

SEQ ID NO: 13 - protein, 97 aa, Homo sapiens
MLGNKRLGLSGLTLALSLLVCLGALAEAYPSKPDNPGEDAPAEDMARYYSALRHYINLITRQRYGKRSSPETLISDLLMRESTENVPRTRLEDPAMW

SEQ ID NO: 14 - protein, 384 aa, Homo sapiens
MNSTLFSQVENHSVHSNFSEKNAQLLAFENDDCHLPLAMIFTLALAYGAVIILGVSGNLALIIIILKQKEMRNVTNILIVNLSFSDLLVAIMCLPFTFVYTLMDHWVFGEAMCKLNPFVQCVSITVSIFSLVLIAVERHQLIINPRGWRPNNRHAYVGIAVIWVLAVASSLPFLIYQVMTDEPFQNVTLDAYKDKYVCFDQFPSDSHRLSYTTLLLVLQYFGPLCFIFICYFKIYIRLKRRNNMMDKMRDNKYRSSETKRINIMLLSIVVAFAVCWLPLTIFNTVFDWNHQIIATCNHNLLFLLCHLTAMISTCVNPIFYGFLNKNFQRDLQFFFNFCDFRSRDDDYETIAMSTMHTDVSKTSLKQASPVAFKKINNNDDNEKI

SEQ ID NO: 15 - protein, 455 aa, Homo sapiens
MSFYSKQDYNMDLELDEYYNKTLATENNTAATRNSDFPVWDDYKSSVDDLQYFLIGLYTFVSLLGFMGNLLILMALMKKRNQKTTVNFLIGNLAFSDILVVLFCSPFTLTSVLLDQWMFGKVMCHIMPFLQCVSVLVSTLILISIAIVRYHMIKHPISNNLTANHGYFLIATVWTLGFAICSPLPVFHSLVELQETFGSALLSSRYLCVESWPSDSYRIAFTISLLLVQYILPLVCLTVSHTSVCRSISCGLSNKENRLEENEMINLTLHPSKKSGPQVKLSGSHKWSYSFIKKHRRRYSKKTACVLPAPERPSQENHSRILPENFGSVRSQLSSSSKFIPGVPTCFEIKPEENSDVHELRVKRSVTRIKKRSRSVFYRLTILILVFAVSWMPLHLFHVVTDFNDNLISNRHFKLVYCICHLLGMMSCCLNPILYGFLNNGIKADLVSLIHCLHM

SEQ ID NO: 16 - protein, 123 aa, Homo sapiens
MARGSALLLASLLLAAALSASAGLWSPAKEKRGWTLNSAGYLLGPHAVGNHRSFSDKNGLTSKRELRPEDDMKPGSFDRSIPENNIMRTIIEFLSFLHLKEAGALDRLLDLPAAASSEDIERS

SEQ ID NO: 17 - protein, 349 aa, Homo sapiens
MELAVGNLSEGNASWPEPPAPEPGPLFGIGVENFVTLVVFGLIFALGVLGNSLVITVLARSKPGKPRSTTNLFILNLSIADLAYLLFCIPFQATVYALPTWVLGAFICKFIHYFFTVSMLVSIFTLAAMSVDRYVAIVHSRRSSSLRVSRNALLGVGCIWALSIAMASPVAYHQGLFHPRASNQTFCWEQWPDPRHKKAYVVCTFVFGYLLPLLLICFCYAKVLNHLHKKLKNMSKKSEASKKKTAQTVLVVVVVFGISWLPHHIIHLWAEFGVFPLTPASFLFRITAHCLAYSNSSVNPIIYAFLSENFRKAYKQVFKCHIRKDSHLSDTKENKSRIDTPPSTNCTHV

SEQ ID NO: 18 - protein, 387 aa, Homo sapiens
MNVSGCPGAGNASQAGGGGGWHPEAVIVPLLFALIFLVGTVGNTLVLAVLLRGGQAVSTTNLFILNLGVADLCFILCCVPFQATIYTLDGWVFGSLLCKAVHFLIFLTMHASSFTLAAVSLDRYLAIRYPLHSRELRTPRNALAAIGLIWGLSLLFSGPYLSYYRQSQLANLTVCHPAWSAPRRRAMDICTFVFSYLLPVLVLGLTYARTLRYLWRAVDPVAAGSGARRAKRKVTRMILIVAALFCLCWMPHHALILCVWFGQFPLTRATYALRILSHLVSYANSCVNPIVYALVSKHFRKGFRTICAGLLGRAPGRASGRVCAAARGTHSGSVLERESSDLLHMSEAAGALRPCPGASQPCILEPCPGPSWQGPKAGDSILTVDVA

SEQ ID NO: 19 - protein, 368 aa, Homo sapiens
MADAQNISLDSPGSVGAVAVPVVFALIFLLGTVGNGLVLAVLLQPGPSAWQEPGSTTDLFILNLAVADLCFILCCVPFQATIYTLDAWLFGALVCKAVHLLIYLTMYASSFTLAAVSVDRYLAVRHPLRSRALRTPRNARAAVGLVWLLAALFSAPYLSYYGTVRYGALELCVPAWEDARRRALDVATFAAGYLLPVAVVSLAYGRTLRFLWAAVGPAGAAAAEARRRATGRAGRAMLAVAALYALCWGPHHALILCFWYGRFAFSPATYACRLASHCLAYANSCLNPLVYALASRHFRARFRRLWPCGRRRRHRARRALRRVRPASSGPPGCPGDARPSGRLLAGGGQGPEPREGPVHGGEAARGPE

SEQ ID NO: 20 - protein, 33 aa, Bos taurus
QPLPDCCRQKTCSCRLYELLHGAGNHAAGILTL

SEQ ID NO: 21 - protein, 131 aa, Homo sapiens
MNLPSTKVSWAAVTLLLLLLLLPPALLSSGAAAQPLPDCCRQKTCSCRLYELLHGAGNHAAGILTLGKRRSGPPGLQGRLQRLLQASGNHAAGILTMGRRAGAEPAPRPCLGRRCSAPAAASVAPGGQSGI

SEQ ID NO: 22 - protein, 425 aa, Homo sapiens
MEPSATPGAQMGVPPGSREPSPVPPDYEDEFLRYLWRDYLYPKQYEWVLIAAYVAVFVVALVGNTLVCLAVWRNHHMRTVTNYFIVNLSLADVLVTAICLPASLLVDITESWLFGHALCKVIPYLQAVSVSVAVLTLSFIALDRWYAICHPLLFKSTARRARGSILGIWAVSLAIMVPQAAVMECSSVLPELANRTRLFSVCDERWADDLYPKIYHSCFFIVTYLAPLGLMAMAYFQIFRKLWGRQIPGTTSALVRNWKRPSDQLGDLEQGLSGEPQPRGRAFLAEVKQMRARRKTAKMLMVVLLVFALCYLPISVLNVLKRVFGMFRQASDREAVYACFTFSHWLVYANSAANPIIYNFLSGKFREQFKAAFSCCLPGLGPCGSLKAPSPRSSASHKSLSLQSRCSISKISEHVVLTSVTTVLP

SEQ ID NO: 23 - protein, 444 aa, Homo sapiens
MSGTKLEDSPPCRNWSSASELNETQEPFLNPTDYDDEEFLRYLWREYLHPKEYEWVLIAGYIIVFVVALIGNVLVCVAVWKNHHMRTVTNYFIVNLSLADVLVTITCLPATLVVDITETWFFGQSLCKVIPYLQTVSVSVSVLTLSCIALDRWYAICHPLMFKSTAKRARNSIVIIWIVSCIIMIPQAIVMECSTVFPGLANKTTLFTVCDERWGGEIYPKMYHICFFLVTYMAPLCLMVLAYLQIFRKLWCRQIPGTSSVVQRKWKPLQPVSQPRGPGQPTKSRMSAVAAEIKQIRARRKTARMLMVVLLVFAICYLPISILNVLKRVFGMFAHTEDRETVYAWFTFSHWLVYANSAANPIIYNFLSGKFREEFKAAFSCCCLGVHHRQEDRLTRGRTSTESRKSLTTQISNFDNISKLSEQVVLTSISTLPAANGAGPLQNW

(19) United States
(12) Patent Application Publication
(10) Pub. No.: US 2005/0186608 A1
(43) Pub. Date: Aug. 25, 2005

(54) Method for the production of transgenic proteins useful in the treatment of obesity and diabetes

(76) Inventor: Byron V. Olsen, Hopkinton, MA (US)

Correspondence Address: GTC BIOTHERAPEUTICS, INC., 175 CROSSING BOULEVARD, SUITE 410, FRAMINGHAM, MA, 01702, US

(21) Appl. No.: 11/060,291

(22) Filed: Feb. 17, 2005

Related U.S. Application Data
(60) Provisional application No. 60/545,790, filed on Feb. 19, 2004.

(51) Int. Cl. C12Q 1/68; C07H 21/04; A61K 38/17; A61K 38/24; C07K 14/705

(52) U.S. Cl. 435/6; 435/69.1; 435/320.1; 435/325; 514/12; 530/350; 536/23.5

(57) ABSTRACT

Transgenic proteins therapeutically useful in the treatment of obesity and related conditions can be produced in and purified from the milk of transgenic animals. The peptides are made as transgenic proteins with a suitable transgenic partner such as human recombinant protein of interest.

### PRIORITY CLAIM

This application claims priority to U.S. Ser. No. 60/545,790, filed on Feb. 19, 2004, the contents of which are incorporated herein by reference.

### FIELD OF THE INVENTION

The present invention relates to the production of obesity related transgenic proteins which are biologically active and can be used to treat obesity and associated pathologies. In particular the current invention provides for the production of Leptin and other anti-aging molecules in the milk of transgenic mammals, particularly non-human placental mammals and provides for the use of such transgenic proteins in therapeutic applications or disease conditions.

### BACKGROUND OF THE INVENTION

As stated above, the present invention relates generally to the field of the transgenic production of transgenic proteins in the milk of transgenic animals. More particularly, it concerns improved methods for generating transgenic proteins capable of therapeutically treating obesity and related pathological conditions.

It is estimated that somewhere between 34 and 61 million people in the U.S. are obese and in much of the developing world this incidence is increasing by about 1% per year. As a general guide, obesity increases the likelihood of death from all causes by 20%, and plays a major role in the development of coronary heart disease, stroke, diabetes and gall bladder disease.

Currently, there are numerous polypeptides, macromolecules and/or proteins (“proteins of interest”) possessing one or more potential therapeutic activities that cannot be exploited pharmaceutically for treatment of obesity and related conditions. There may be various reasons for this inability, such as low stability in vivo, altered glycosylation patterns found in proteins from non-eukaryotic cells, improper translational processing, difficult in vitro secretion, tertiary structure, transmembrane sequences making expression difficult, immunogenicity, difficulty in producing recombinant proteins on an industrially acceptable scale or the like. Moreover, some therapeutically valuable proteins do not give the expected results in vivo because of problems related to the method of their purification, administration or pharmacokinetics.

The present invention makes it possible to overcome these disadvantages with regard to the production of molecules effective in the treatment of obesity and related conditions. The instant invention provides for the production of specific molecules transgenically, permitting the exploitation of the physiological properties or effects of the proteins of interest. The present invention results especially from the demonstration that it is possible to express both transmembrane proteins and proteins with complex tertiary structures in high volume and to use them singly or together to achieve synergistic therapeutic effects.

Signals from the brain play critical roles in the regulation of body weight and metabolism. Information about nutrient stores, satiety, palatability of food, etc., are communicated to the brain by various endocrine hormones, nutrient/metabolite signals or by neural pathways that connect the brain and the rest of the body. The cognitive and limbic centers of the brain also impact the quantity and type of food consumed. Each of these inputs is integrated by the brain and ultimately translated into appropriate compensatory changes in food intake and/or energy expenditure. The brain is responsible for the integration of each of these inputs to form the final response. This response is then mediated via the activation of discrete neurotransmitter and neuropeptide signaling pathways by the brain. While the complex neural circuitry that regulates body weight, obesity and many related pathologies is not fully defined, a number of specific neurotransmitters, proteins and neuropeptides have been implicated in the process. The difficulty in developing a rational approach to exploiting these specific drug targets has been both in the production of proteins retaining sufficient physiological activity so as to be useful in needed therapeutic treatments for weight loss and in determining what combinations of drugs can effectuate a beneficial synergistic effect.

The transgenic proteins produced according to the current invention make it possible to accelerate weight loss and/or reduce caloric intake thereby reducing the detrimental effects of obesity and ameliorating the onset of negative pathologies such as Type II diabetes. Certain of the proteins of interest according to the current invention may also be expressed secreted by recombinant organisms, such as in cell culture production facilities, or transgenic mammals, at levels permitting their commercial exploitation. Along this line, transgenic mammals are a preferred manufacturing and expression vehicle for the transgenic proteins of the invention.

In a preferred embodiment the current invention provides for the bulk production of obesity related transgenic proteins of interest in the milk of transgenic mammals. The production of a transgenic protein of interest in milk is ideal as a bulk process because very large volumes of milk that can be produced, collected and purified using known dairy technology. A second advantage of using a transgenic mammalian process is that some reactions which can be essential for biological activity in humans, for example carboxy-terminal amidation, the development of cysteine-cysteine bonds for di-sulfide bridges and post-translational modifications are difficult or impossible to develop reliably and in good yield by currently available chemical means, or through bacterial or other in vitro techniques. For example, carboxy-terminal amidation is catalyzed by a specific enzyme which recognizes and modifies a transgenic protein of interest or proteins with a glycine residue at the carboxy terminus. Therefore, the transgenic proteins of the invention can be specifically manipulated before secretion into the milk of transgenic animals to enhance physiological activity. This is only one example of a range of post-translational modifications which can be carried out only by the biosynthetic pathways in the mammary gland and which can potentially be harnessed for the synthesis of particular transgenic proteins. Other examples of desirable post-translational modifications include: γ-carboxylation of glutamic acid residues and the addition of O- and N-linked glycosylation. In essence the glycosylation of proteins from a whole animal mammalian system are superior in the development of non-immunogenic molecules of interest because the patterns of glycosylation are much closer to those of the in vivo mammalian molecules than those produced by bacterial, yeast, or even mammalian in vitro cell culture. This means that the proteins expressed by the preferred embodiment of the current invention have a similar sugar profile to naturally occurring molecules therefore limiting immunogenicty and antigenicity problems often encountered with recombinant products derived from cell culture sources.

According to the prior art, the generation of an animal capable of producing a recombinant protein of interest is known. However, what remained unknown prior to the current invention was the level of genetic manipulation required for the current invention, the modified sequences of the various transgenic protein components available, the synergistic effect of the current obesity related molecules one to another, the positive beneficial effect of making certain transgenically produced proteins structurally similar but physiologically non-functional, the actual physiological effect of certain of the proteins and peptides described herein and the disease states or pathologies in which they are useful.

The discovery of leptin set in motion an intense research effort to understand the genetic, hormonal, neurochemical and behavioral basis of obesity and body weight regulation in humans and the fruits of this effort are now becoming evident. Several proteins, neuropeptides and neurotransmitters, including the PYY peptide, that are involved in the central control of bodyweight have been identified and have been shown to be mediators of the effects of leptin on body weight. Accordingly, the new processes of the current invention as well as more efficient methods of treatment, formulation and production are needed to treat the growing incidence of obesity and its associated pathologies.

### SUMMARY OF THE INVENTION

Briefly stated, the current invention provides a method for the production of obesity related transgenic proteins of interest, preferably through the use of transgenic animals. The method involves transfecting a non-human mammalian cell-line with a given transgene construct, the construct containing at least one recombinant DNA coding sequence encoding a desired peptide sequence that retains the biological activity of an individual protein of interest or selectively and purposely disrupts it. The process involves developing the DNA construct; selecting a cell line(s) in which the desired recombinant sequence has been inserted into the genome of that cell or cell-line; performing a nuclear transfer procedure to generate a transgenic animal heterozygous for the desired transgenic protein. Thereafter the transgenic protein expressing the obesity related transgenic protein may be collected from the milk or other bodily fluid of the transgenic animal and purified for use as a therapeutic agent.

An additional step that may be performed according to the invention is to biopsy the heterozygous transgenic animal. Thereafter, according to the current invention the cell line can be expanded in vitro with the biopsied cell-line obtained from the heterozygous animal used to develop multiple transgenic animals in a shorter time period.

Alternatively or in addition to, a nuclear transfer procedure can be conducted to generate a mass of transgenic cells useful for research, serial cloning, or other in vitro use. In a preferred embodiment of the current invention surviving cells are characterized by one of several known molecular biology methods including without limitation FISH, Southern Blot, or PCR. The methods provided above will allow for the accelerated production of a transgenic herd of animals homozygous for desired transgene(s) and thereby the more efficient production of a desired biopharmaceutical. In this way the current invention allows for the production of genetically desirable livestock or non-human mammals themselves expressing an obesity related transgenic protein of interest.

One subject of the present invention therefore relates to obesity related transgenic proteins containing an active protein and/or a dysfunctional receptore that is normally part of the signal transduction pathway controlled by the brain that affects hunger, fat storage, and/or energy expenditure. One preferred embodiment of the current invention is the use of multiple transgenically derived molecules to treat obesity or related pathologies that achieve synergistic effects when used together.

Another subject of the invention relates to a process for preparing the chimeric molecules described above. More specifically, this process consists in causing a eukaryotic or prokaryotic cellular host to express a nucleotide sequence encoding the desired transgenic protein, and then in harvesting the transgenic protein product.

Accordingly, it is an object of the invention to provide a pharmaceutical agent that is capable of treating obesity.

Another aspect of this invention is directed to a method for treating abnormal insulin release comprising administering to a mammal (e.g., a female or male human) a therapeutically effective amount of a transgenic protein of interest, a prodrug thereof, or a pharmaceutically acceptable salt of said compound or of said prodrug.

Another aspect of this invention is directed to a method for treating insulin resistance comprising administering to a mammal (e.g., a female or male human) a therapeutically effective amount of a transgenic protein of interest, a prodrug thereof, or a pharmaceutically acceptable salt of said compound or of said prodrug.

Another aspect of this invention is directed to a method for treating impaired glucose tolerance comprising administering to a mammal (e.g., a female or male human) a therapeutically effective amount of a transgenic protein of interest, a prodrug thereof, or a pharmaceutically acceptable salt of said compound or of said prodrug.

Another aspect of this invention is directed to a method for treating Type II diabetes mellitus comprising administering to a mammal (e.g., a female or male human) a therapeutically effective amount of a transgenic protein of interest, a prodrug thereof, or a pharmaceutically acceptable salt of said compound or of said prodrug.

Another aspect of this invention is directed to a method for treating Type II diabetes mellitus comprising administering to a mammal (e.g., a female or male human) a therapeutically effective amount of a transgenic protein of interest, a prodrug thereof, or a pharmaceutically acceptable salt of said compound or of said prodrug in addition to a modified lower dosing of insulin via pump means.

Another aspect of this invention is directed to a method for treating Type I diabetes comprising administering to a mammal (e.g., a female or male human) a therapeutically effective amount of a transgenic protein of interest, a prodrug thereof, or a pharmaceutically acceptable salt of said compound or of said prodrug.

Another aspect of this invention is directed to a method for treating hypertension comprising administering to a mammal (e.g., a female or male human) a therapeutically effective amount of a transgenic protein of interest, a prodrug thereof, or a pharmaceutically acceptable salt of said compound or of said prodrug.

Another aspect of this invention is directed to a method for treating hyperphagia comprising administering to a mammal (e.g., a female or male human) a therapeutically effective amount of a transgenic protein of interest, a prodrug thereof, or a pharmaceutically acceptable salt of said compound or of said prodrug.

Another aspect of this invention is directed to a method for treating gall stones comprising administering to a mammal (e.g., a female or male human) a therapeutically effective amount of a transgenic protein of interest, a prodrug thereof, or a pharmaceutically acceptable salt of said compound or of said prodrug.

Another aspect of this invention is directed to a method for treating cardiovascular disease comprising administering to a mammal (e.g., a female or male human) a therapeutically effective amount of a transgenic protein of interest, a prodrug thereof, or a pharmaceutically acceptable salt of said compound or of said prodrug.

Another aspect of this invention is directed to a method for treating hyperlipidemia comprising administering to a mammal (e.g., a female or male human) a therapeutically effective amount of a transgenic protein of interest, a prodrug thereof, or a pharmaceutically acceptable salt of said compound or of said prodrug.

This invention is also directed to pharmaceutical compositions which comprise an amount of a transgenic protein of interest, a prodrug thereof, or a pharmaceutically acceptable salt of said compound or of said prodrug and a pharmaceutically acceptable vehicle, diluent or carrier.

This invention is also directed to pharmaceutical compositions for the treatment of obesity which comprise an obesity treating amount of a transgenic protein of interest, a prodrug thereof, or a pharmaceutically acceptable salt of said compound or of said prodrug and a pharmaceutically acceptable vehicle, diluent or carrier.

These and other objects which will be more readily apparent upon reading the following disclosure may be achieved by the present invention.

### BRIEF DESCRIPTION OF THE DRAWINGS

FIG. 1 Shows a flowchart of the methods involved in practicing the invention.

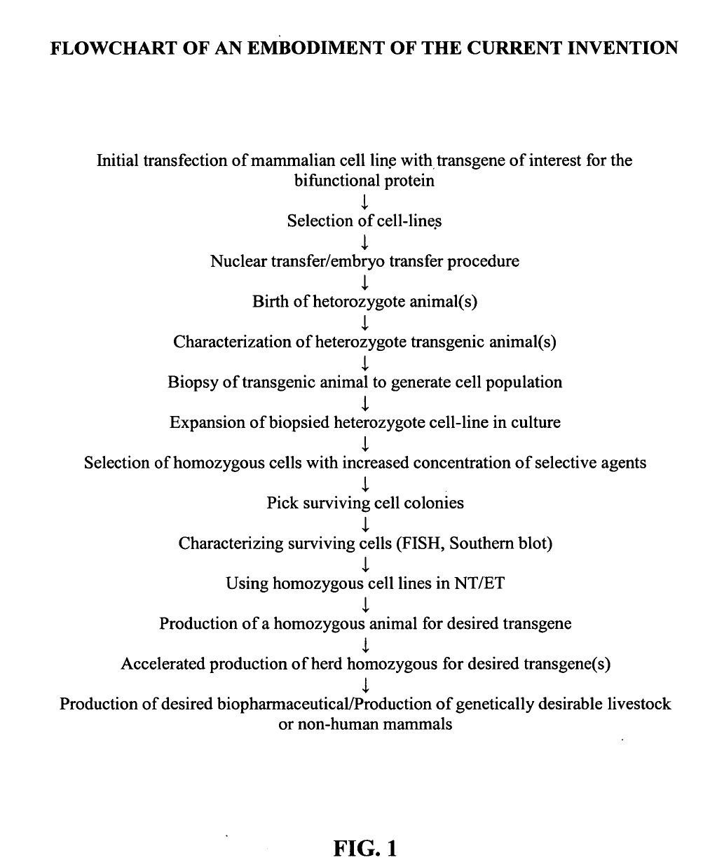

FIG. 2 Shows a Generalized Diagram of the Process of Creating Cloned Animals through Nuclear Transfer.

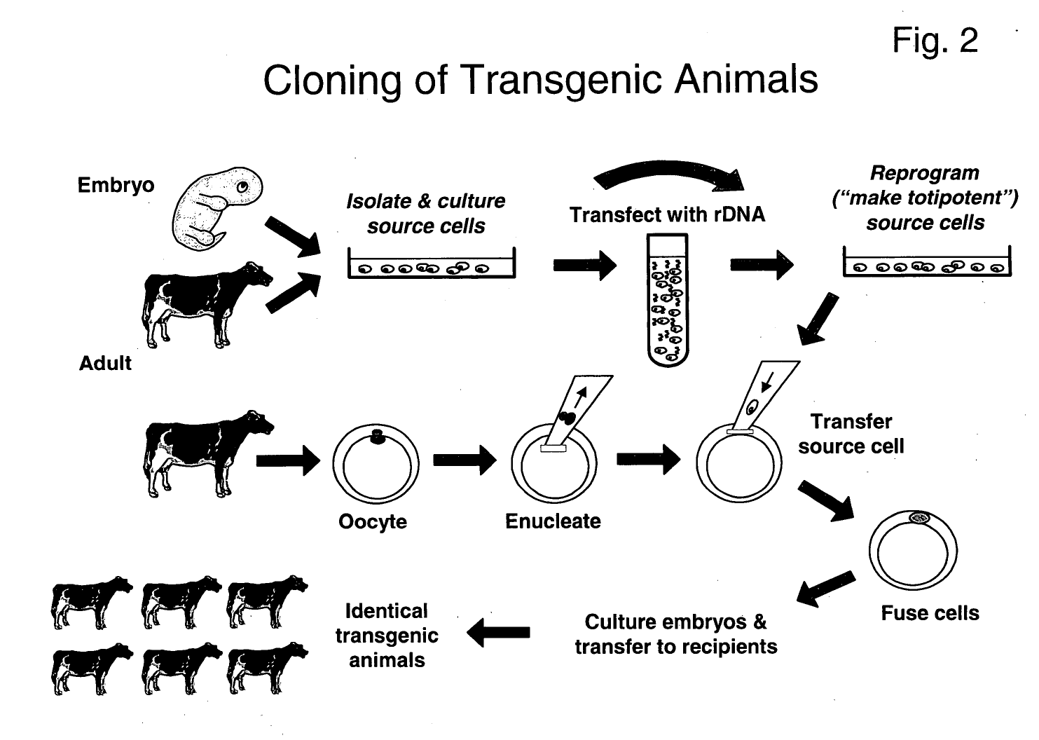

### DETAILED DESCRIPTION

The following abbreviations have designated meanings in the specification:

Abbreviation Key:

| | |
|---|---|
| Somatic Cell Nuclear Transfer | (SCNT) |
| Nuclear Transfer | (NT) |
| Synthetic Oviductal Fluid | (SOF) |
| Fetal Bovine Serum | (FBS) |
| Polymerase Chain Reaction | (PCR) |
| Bovine Serum Albumin | (BSA) |

Explanation of Terms:
Bovine—Of or relating to various species of cows.
Biological Fluid—an aqueous solution produced by an organism, such as a mammal, bird, amphibian, or reptile, which contains proteins that are secreted by cells that are bathed in the aqueous solution. Examples include: milk, urine, saliva, seminal fluid, vaginal fluid, synovial fluid, lymph fluid, amniotic fluid, blood, sweat, and tears; as well as an aqueous solution produced by a plant, including, for example, exudates and guttation fluid, xylem, phloem, resin, and nectar.
Biological-fluid producing cell—A cell that is bathed by a biological fluid and that secretes a protein into the biological fluid.
Biopharmaceutical—shall mean any medicinal drug, therapeutic, vaccine or any medically useful composition whose origin, synthesis, or manufacture involves the use of microorganisms, recombinant animals (including, without limitation, chimeric or transgenic animals), nuclear transfer, microinjection, or cell culture techniques.
Caprine—Of or relating to various species of goats.
Encoding—refers generally to the sequence information being present in a translatable form, usually operably linked to a promoter (e.g., a beta-casein or beta-lacto globulin promoter). A sequence is operably linked to a promoter when the functional promoter enhances transcription or expression of that sequence. An anti-sense strand is considered to also encode the sequence, since the same informational content is present in a readily accessible form, especially when linked to a sequence which promotes expression of the sense strand. The information is convertible using the standard, or a modified, genetic code.
Expression Vector—A genetically engineered plasmid or virus, derived from, for example, a bacteriophage, adenovirus, retrovirus, poxvirus, herpesvirus, or artificial chromosome, that is used to transfer an obesity related transgenic protein coding sequence, operably linked to a promoter, into a host cell, such that the encoded recombinant obesity related transgenic protein is expressed within the host cell.
Functional Proteins—Proteins which have a biological or other activity or use, similar to that seen when produced endogenously.
Transgenic Slide—A glass slide for parallel electrodes that are placed a fixed distance apart. Cell couplets are placed between the electrodes to receive an electrical current for transgenic and activation.
Homologous Sequences—refers to genetic sequences that, when compared, exhibit similarity. The standards for homology in nucleic acids are either measures for homology generally used in the art or hybridization conditions. Substantial homology in the nucleic acid context means either that the segments, or their complementary strands, when compared, are identical when optimally aligned, with appropriate nucleotide insertions or deletions, in at least about 60% of the residues, usually at least about 70%, more usually at least about 80%, preferably at least about 90%, and more preferably at least about 95 to 98% of the nucleotides. Alternatively, substantial homology exists when the segments will hybridize under selective hybridization conditions, to a strand, or its complement. Selectivity of hybridization exists when hybridization occurs which is more selective than total lack of specificity. Typically, selective hybridization will occur when there is at least about 55% homology over a stretch of at least about 14 nucleotides, preferably at least about 65%, more preferably at least about 75%, and most preferably at least about 90%.
Leader sequence or a “signal sequence”—a nucleic acid sequence that encodes a protein secretory signal, and, when operably linked to a downstream nucleic acid molecule encoding a transgenic protein and directs secretion. The leader sequence may be the native human leader sequence, an artificially-derived leader, or may obtained from the same gene as the promoter used to direct transcription of the transgene coding sequence, or from another protein that is normally secreted from a cell.
Milk-producing cell—A cell (e.g., a mammary epithelial cell) that secretes a protein into milk.
Milk-specific promoter—A promoter that naturally directs expression of a gene in a cell that secretes a protein into milk (e.g., a mammary epithelial cell) and includes, for example, the casein promoters, e.g., α-casein promoter (e.g., alpha S-1 casein promoter and alpha S2-casein promoter), β-casein promoter (e.g., the goat beta casein gene promoter (DiTullio, BIOTECHNOLOGY 10:74-77, 1992), γ-casein promoter, and κ-casein promoter; the whey acidic protein (WAP) promoter (Gorton et al., BIOTECHNOLOGY 5: 1183-1187, 1987); the β-lactoglobulin promoter (Clark et al., BIOTECHNOLOGY 7: 487-492, 1989); and the α-lactalbumin promoter (Soulier et al., FEBS LETTS. 297:13, 1992). Also included are promoters that are specifically activated in mammary tissue and are thus useful in accordance with this invention, for example, the long terminal repeat (LTR) promoter of the mouse mammary tumor virus (MMTV).
Nuclear Transfer—This refers to a method of cloning wherein the nucleus from a donor cell is transplanted into an enucleated oocyte.
Operably Linked—A gene and one or more regulatory sequences are connected in such a way as to permit gene expression when the appropriate molecules (e.g., transcriptional activator proteins) are bound to the regulatory sequences.
Ovine—Of or relating to or resembling sheep.
Parthenogenic—The development of an embryo from an oocyte without the penetration of sperm.
Pharmaceutically Pure—This refers to transgenic protein that is suitable for unequivocal biological testing as well as for appropriate administration to effect treatment of a human patient. Substantially pharmaceutically pure means at least about 90% pure.
Porcine—of or resembling pigs or swine.
Promoter—A minimal sequence sufficient to direct transcription. Also included in the invention are those promoter elements which are sufficient to render promoter-dependent gene expression controllable for cell type-specific, tissue-specific, temporal-specific, or inducible by external signals or agents; such elements may be located in the 5′ or 3′ or intron sequence regions of the native gene.
Protein—as used herein is intended to include glycoproteins, as well as proteins having other additions. This also includes fragmentary or truncated polypeptides that retain physiological function.
Recombinant—refers to a nucleic acid sequence which is not naturally occurring, or is made by the artificial combination of two otherwise separated segments of sequence. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functional polypeptide sequences to generate a single genetic entity comprising a desired combination of functions not found in the common natural forms. Restriction enzyme recognition sites are often the target of such artificial manipulations, but other site specific targets, e.g., promoters, DNA replication sites, regulation sequences, control sequences, or other useful features may be incorporated by design. A similar concept is intended for a recombinant, e.g., a obesity related transgenic protein according to the instant invention.
Therapeutically-effective amount—An amount of a therapeutic molecule or a fragment thereof that, when administered to a patient, inhibits or stimulates a biological activity modulated by that molecule.
Transformation, “Transfection,” or “Transduction”—Any method for introducing foreign molecules into a cell. Lipofection, DEAE-dextran-mediated transfection, microinjection, nuclear transfer (see, e.g., Campbell et al. BIOL. REPROD. 49:933-942, 1993; Campbell et al., NATURE 385:810-813, 1996), protoplast transgenic, calcium phosphate precipitation, transduction (e.g., bacteriophage, adenoviral retroviral, or other viral delivery), electroporation, and biolistic transformation are just a few of the methods known to those skilled in the art which may be used.
Transformed cell or Transfected cell—A cell (or a descendent of a cell) into which a nucleic acid molecule encoding obesity related has been introduced by means of recombinant DNA techniques. The nucleic acid molecule may be stably incorporated into the host chromosome, or may be maintained episomally.
Transgene—Any piece of a nucleic acid molecule that is inserted by artifice into a cell, or an ancestor thereof, and becomes part of the genome of the animal which develops from that cell. Such a transgene may include a gene which is partly or entirely exogenous (i.e., foreign) to the transgenic animal, or may represent a gene having identity to an endogenous gene of the animal.
Transgenic—Any cell that includes a nucleic acid molecule that has been inserted by artifice into a cell, or an ancestor thereof, and becomes part of the genome of the animal which develops from that cell.
Transgenic Organism—An organism into which genetic material from another organism has been experimentally transferred, so that the host acquires the genetic information of the transferred genes in its chromosomes in addition to that already in its genetic complement.
Ungulate—of or relating to a hoofed typically herbivorous quadruped mammal, including, without limitation, sheep, swine, goats, cattle and horses.
Vector—As used herein means a plasmid, a phage DNA, or other DNA sequence that (1) is able to replicate in a host cell, (2) is able to transform a host cell, and (3) contains a marker suitable for identifying transformed cells.

According to the present invention, there is provided a method for the production of a transgenic protein of interest, the process comprising expressing in the milk of a transgenic non-human placental mammal a transgenic protein useful in the treatment of obesity or related pathologies. The term “treating”, “treat” or “treatment” as used herein includes preventative (e.g., prophylactic) and palliative treatment.

Transgenic Leptin

Characterized in 1994 (Zhang et al, NATURE) as the product of the ob gene, Leptin is a 16 kDa protein (167 aa) of the cytokine family expressed in adipose and other tissues. In one experimental model the leptin gene has been mutated to produce ob/ob obese mice, in another the leptin receptor gene is mutated in db/db obese mice. Again clearly demonstrating the involvement of this molecule in the body levels of fat tissues. That is, it is important to note that leptin levels in plasma reflect size of fat tissue—that decrease during starvation and increase in obesity. The leptin receptor was first cloned in 1995 by Tartaglia et al., (CELL 83, 1263). The ob/ob mice are also characterized by hypogonad-otropichypogonadism, a condition that results in sterility. In addition, ob/ob mice are clinically pre-pubertal, with serum concentrations of luteinizing hormone, follicle-stimulating hormone, oestradiol and testosterone all at pre-pubertal levels. Moreover, these ob/ob mice are markedly hypercortisolaemic with insulin levels being consistently elevated after feeding, consistent with hyperinsulinaemia, similar phenotypes are seen in many overweight humans.

Human and murine leptin proteins possess over 80% amino acid homology. Leptin is an integral part of a feedback-loop that communicates information about fat stores to the brain. Leptin secreted into the circulation acts on a specific receptor in the hypothalamus to actually decrease food intake while increasing energy expenditure. Mutations in the leptin gene result in complete deficiency of circulating plasma leptin and severe obesity in the ob/ob mouse. Mutations in the leptin receptor, a member of the cytokine receptor family, are responsible for the obese phenotype of the db/db mouse and the Zucker fatty rat. Interestingly, most obese humans have much higher plasma levels of leptin than non-obese humans, suggesting that most obesity is associated with leptin resistance rather than leptin deficiency. However, 5-10% of obese humans have low plasma leptin levels and mutations of the leptin and leptin receptor genes have been noted within them that may lead or contribute to leptin deficiency or leptin resistance obesity. Therefore, according to the instant invention leptin can be utilized as therapeutic treatment for obesity and related conditions in humans. Medical studies exist to show that the administration of leptin produces reductions in body weight and may be useful in the treatment of leptin deficiency. Nevertheless, a leptin receptor agonist could be a very effective therapeutic compound. Alternatively, a defective leptin molecule or defective leptin receptor could be useful a therapeutic compound in those situations in which weight gain is desired—such as in anorexia nervosa. Below is the Genbank/EMBL/DDBJ reference for the amino acid sequence of human Leptin which can be produced transgenically by means of the instant invention.
Seq. Id.: 1 Genbank/EMBL/DDBJ Accession No. BAA09787, from the National Center for Biotechnology Information—the amino acid sequence of human Leptin (1-167 amino acid residues).
   Ghrelin

Growth hormone (GH), produced and released from the anterior pituitary, controls body growth, carbohydrate-protein-lipid metabolism and water-electrolyte balance. In this sense, Ghrelin is an acyl-peptide gastric hormone acting on the pituitary and hypothalamus to stimulate growth hormone (GH) release, adiposity, and appetite. Ghrelin endocrine activities are entirely dependent on its acylation state and are mediated by GH secretagogue (GHS) receptor (GHSR)-1a, a G protein-coupled receptor mostly expressed in the pituitary and hypothalamus, previously identified as the receptor for a group of synthetic molecules featuring GH secretagogue (GHS) activity.

It is known that GH release is stimulated by hypothalamic GH-releasing hormone (GHRH) and inhibited by somatostatin. GH secretagogues (“GHS's”) are synthetic compounds that are potent stimulators of GH release. The G-protein-coupled receptor GHS receptor (“GHS-R”), has emerged as a strong regulator of GH release. Until recently, the identity of an endogenous ligand for GHS-R was unknown. The molecule now so identified is Ghrelin and has been purified and identified from rat and human stomach. As expected this molecule is now known to act as a ligand for the GHS-R.

More specifically, Ghrelin is a newly discovered gastric 28 amino-acid peptide which stimulates feeding and antagonizes leptin's anorexic effects. Ghrelin promotes growth hormone secretion and exerts effects on neuroendocrine systems regulating appetite and food intake. Moreover, Ghrelin has been reported to influence insulin secretion and glucose metabolism. It is known that Ghrelin has a negative impact on insulin levels and may be implicated in the development of type 2 diabetes. Ghrelin concentrations in blood are reduced in obese humans compared to lean control subjects, but whether this is cause or effect is not defined. Patients with anorexia nervosa have higher than normal plasma Ghrelin levels, which decrease if weight gain occurs.

In the prior art, the i.v. administration of ghrelin above a minimally active dose (10 pmol) to free-feeding rats increased food intake in a dose-dependent manner. A GHSR antagonist suppressed ghrelin-induced feeding and furthermore, the administration of ghrelin-specific antibodies suppressed starvation-induced feeding in a dose-dependent manner, suggesting that ghrelin is a powerful, endogenous orexigenic peptide. In humans, IV bolus injection or infusion of ghrelin induces hunger.

Unlike ghrelin, most other hypothalamic peptides—for example, neuropeptide Y (NPY), agouti-related peptide (AGRP), orexins, melanin-concentrating hormone (MCH), and galanins—that stimulate feeding when administered centrally are ineffective when administered into the periphery. Ghrelin is the first identified circulating hormone that promotes feeding following systemic administration. In humans, plasma ghrelin levels rise sharply before and fall shortly after every meal. These findings indicate that ghrelin may serve as an indicator of short-term energy balance and might be a candidate for a meal-initiation signal. Moreover, given the putative difference in mode of action or chain of action, Ghrelin antagonists when administered with other weight loss promoting proteins could have a substantial synergistic effect with one or more of these molecules.

Prader-Willi syndrome is another disorder potentially relevant to the Ghrelin's physiological activity. Affected patients develop extreme obesity associated with uncontrollable and voracious appetite. The plasma Ghrelin levels are exceptionally high in comparison to patients similarly obese due to other causes. Prader-Willi syndrome is clearly a complex disease with many defects but it may be that excessive Ghrelin production contributes to the appetite and obesity components.

Rat and human mature Ghrelin (28-aa) are produced from a 117 amino acid precursor peptide that has an initial signal peptide followed by the mature Ghrelin sequence. At the C-terminus of the mature cleaved Ghrelin sequence there is a Proline-Arginine set of residues that affects processing of the molecule. In the rat stomach, two isoforms of mRNA for pro-Ghrelin sequences have been identified and are produced from the gene by alternative splicing mechanisms. One mRNA encodes the Ghrelin precursor, and another encodes an alternate Ghrelin precursor. The secondary Ghrelin precursor (designated “des-Gln14-Ghrelin” in the literature) is identical to Ghrelin, except for the deletion of a single residue Gln14 but has no known physiological function. This deletion results from the use of the C-A-G codon, which encodes Gln14 as a splicing signal. The secondary Ghrelin precuresor is only present in low amounts in the stomach, indicating that the first mentioned Ghrelin precursor is the more important form. Ghrelin has an unusual modification at its Serine 3 residue that is N-octanoylated and this modification appears to be essential for normal biological activity. Ghrelin is the first known example of a bioactive peptide modified by an acyl acid.

There is no structural homology between Ghrelin and peptide GHS's (GHRP-6 or hexarelin). Ghrelin has partial sequence homology with motilin. Rat Ghrelin is expressed in the stomach, small and large intestine, and brain regions (hypothalamic arcuate nucleus) that are involved the regulation of food intake. Normal adult human plasma samples contain 100-120 fmol Ghrelin/ml. Both Ghrelin and GHS-R expression have been seen in the heart, suggesting that Ghrelin might have some cardiovascular effects or indications. Ghrelin administration stimulates GH secretion but also causes weight gain by increasing food intake and reduction in fat utilization.

Plasma Ghrelin concentrations are not affected by diabetic status, but decline with age and may be regulated by insulinemia. Obesity and leptinemia do not appear to have an impact on plasma Ghrelin concentration independent of insulinemia. The physiologically interplay between insulin and Ghrelin therefore contributes to food intake and body weight regulation. For the purposes of the current invention it is preferable to express a dysfunctional Ghrelin Receptor that would have a beneficial therapeutic effect in weight reduction and related pathologies when administered to a patient by attaching to and capturing plasma Ghrelin thereby eliminating or reducing its physiological activity. In this sense the use of dysfunctional Ghrelin receptor would be to pull out of circulation as much Ghrelin as possible. In a preferred embodiment of the invention a dysfunctional Ghrelin receptor compound would be administered with an amount of leptin to enhance weight-loss in a synergistic way.
Seq. Id.: 2 Genbank/EMBL/DDBJ Accession No. AAQ89412, from the National Center for Biotechnology Information—the amino acid sequence of Ghrelin (1-177 amino acid residues).
Seq. Id.: 3 Genbank/EMBL/DDBJ Accession No. Q92847, from the National Center for Biotechnology Information—the amino acid sequence of the Ghrelin Receptor (GHS-R) Isoforms A and B. (both of which have 366 amino acid residues).
   Glucagon

The pancreatic peptide glucagon, is secreted by the alpha cells of the islets of Langerhans in order to maintain plasma glucose concentrations during fasting. It is produced as a pro-hormone (pro-glucagon) and gains activity after cleavage. The biological action of glucagon is exerted upon target tissue following receptor binding and adenylyl cyclase activation. Glucagon binds to glucagon receptors on liver cells, triggering degradation of glycogen and release of glucose into the blood. The glucagon receptor is a G protein-coupled receptor. Diabetes mellitus, a disease of aberrant carbohydrate metabolism, is manifested by a tremendous elevation in plasma glucose levels.

Glucagon is a catabolic enzyme via its antagonism of insulin activity through a relatively simple biochemical pathway. Glucagon acts on the liver where it induces glycogenolysis and gluconeogenesis thus increasing plasma glucose. In type 2 diabetes the insulin/glucagon ratio is significantly decreased. Therefore inhibition of glucagon action in these patients may improve hyperglycemia by inhibition of hepatic glucose production from glycogenolysis and gluconeogenesis. Glucagon has a short plasma half-life (<3 min.) in vivo. Renal extraction is a major factor limiting the metabolic stability, but the mechanisms responsible remain poorly understood in the prior art. For the purposes of the current invention the use of a dysfunctional glucagon receptor as expressed and secreted by transgenic animals could be used to treat anorexia nervosa or similar conditions while conditions associated with diabetes and heightened insulin resistance could be treated with the increased availability and use of recombinant glucagon.

The glucagon receptor is expressed in liver, pancreatic β-cells, kidney, adipose tissue, heart, and in some regions of the central nervous system. A decrease in receptor activity in vivo via delivery of an exogenous defective or dysfunctional receptor may affect fat deposition and catabolism by diverse mechanisms. A reduced cyclic-adenosine monophosphate-mediated lypolitic response or, alternatively, a deficiency of glucagon counter-regulatory activity could potentially lead to an increase of the anabolic action of insulin useful in the treatment of obesity and related conditions.
Seq. Id.: 4 Genbank/EMBL/DDBJ Accession No. AAP35459, from the National Center for Biotechnology Information—the amino acid sequence of human Glucagon (1-180 amino acid residues).
Seq. Id.: 5 Genbank/EMBL/DDBJ Accession No. NP 000151, from the National Center for Biotechnology Information—the amino acid sequence of the human Glucagon Receptor. (which has 1-477 amino acid residues).
   PYY

Peptide YY (PYY) is a 36 amino acid peptide secreted by intestinal L-cells in response to nutrients. It is known to regulate a number of digestive functions such as gastric emptying, gastrointestinal motility, exocrine pancreatic secretion and gastric acid secretion. The subpeptide PYY, containing residues 3-36 of the normal peptide, is produced through cleavage of PYY by the enzyme dipeptidyl peptidase IV and constitutes a significant proportion of circulating PYY-like immunoreactivity. It has been demonstrated that the continuous consumption by laboratory animals of subpeptide PYY in Zucker Diabetic Fatty (ZDF) rats reduces food intake but not body weight gain.

The physiological presence of subpeptide PYY in ZDF rats resulted in a dose-dependent improvement in glycemic control via an as yet unidentified mechanism. However, this mechanism is likely associated with changes in nutrient uptake, and not directly associated with changes in insulin and glucagon secretion.
Seq. Id.: 6 Genbank/EMBL/DDBJ Accession No. NP 004151, from the National Center for Biotechnology Information—the amino acid sequence of the human PYY peptide (which has 1-97 amino acid residues).
   ORIGIN
   1 mvfvrrpwpa lttvllallv clgalvdayp ikpeapgeda speelnryya slrhylnlvt 61 rqrygkrdgp drllsktffp dgedrpvrsr segpdlw
   (PYY fragment being amino acid residues 3-36 of the above).
      Melanocortin

The melanocortins are peptides derived from the propiomelanocortin (POMC) polypeptide precursor—α-melanocyte stimulating hormone (“α-MSH”). There are five known melanocortin receptors (MC-R 1-5), of which MC3-R and MC4-R are found in rat hypothalami. The α-MSH moleucule is an agonist against all of the MC-R's. The agouti-related protein (AGRP) is present naturally in the hypothalamus and is a competitive antagonist at the MC3 and MC4-R's. There is considerable evidence to indicate that melanocortin peptide (“MC”) has a substantial role in the regulation of food intake and overall bodyweight of individuals. The melanocortin peptide family, which includes, α-MSH, β-MSH, γ-MSH and ACTH are all post-translationally processed products of the propio-melanocortin (POMC) gene. These peptides mediate their physiological effects by interacting with at least five structurally related G protein-coupled receptors designated in the literature as MC1 through MC5. The genetically obese agouti mouse develops late onset obesity due to ectopic over-expression of the agouti signaling protein (ASP), a protein that is a high affinity antagonist of the MC 1 and MC4 receptors, respectively. AGRP is structurally related protein, is expressed in the brain and acts as an antagonist of the MC3 and MC4 receptors. A C-terminal fragment of AGRP, utilizing AGRP residues 83-132, retains the biological activity of the full length protein. Over expression of AGRP in transgenic mice in turn leads to obesity.

According to an embodiment of the current invention the AGRP functions as an endogenous melanocortin receptor antagonist that regulates central melanocortin neurotransmissions. Humans that cannot synthesize melanocortin peptides or that have mutations in the MC4 receptor gene, rendering these peptides non-active tends towards obesity. Taken together, these facts indicate that the activation of the MC4 receptor by melanocortin peptides will restrain weight gain and that therefore MC4 receptor agonists might be useful anti-obesity agents.

MC peptides are cleaved from the precursor polypeptide: proopiomelanocortin, which is itself synthesized in the arcuate nucleus of the hypothalamus and brainstem. The α-Melanocyte-stimulating hormone (α-MSH) is an MC peptide that acts as an endogenous agonist of MC3 and 4 receptor subtypes (MC3/4-R). Through this action, α-MSH provides an inhibition of food intake that constrains body weight gain. This role for MSH is evidenced in its ability to reduce food intake for up to 48 hours in rats when centrally injected, a property shared with MTII (MC3/4-R agonist), a synthetic ligand of MSH. Dependency on the MC4-R for these effects is supported by the inability of MTII to reduce intake in MC4-R-deficient mice, which are hyperphagic and obese.

The actions of α-MSH on MC3/4-R's and food intake are potently antagonized at these receptors through the presence of agouti protein and the structurally related agouti-related peptide (AGRP), a 132-amino acid peptide that is synthesized exclusively in the arcuate nucleus of the hypothalamus.

The importance of MC in the energy homeostasis of the body is strongly suggested by the ability of MC3/4-R antagonism to induce hyperphagia and obesity. Notably, the overexpression of either agouti (Ay mice) or AGRP results in phenotypes that include obesity, hyperphagia, and hyperinsulinemia, and this phenotype is recapitulated in mice deficient in MC4-R. In mouse models of obesity caused by leptin deficiency (ob/ob) or leptin receptor dysfunction (db/db), an 8- to 10-fold elevation in hypothalamic AGRP mRNA is found. In normal but 48-h fasted mice, when leptin levels would predictably be decreased, AGRP mRNA is observed to reach 10- to 15-fold elevations.
Seq. Id.: 7 Genbank/EMBL/DDBJ Accession No. NM 00939, from the National Center for Biotechnology Information—the mRNA sequence of the human Homo sapiens proopiomelanocortin (adrenocorticotropin/beta-lipotropin/alpha-melanocyte stimulating hormone/beta-melanocyte stimulating hormone/beta-endorphin) (POMC), containing α-MSH. (which has 1-1071 base pairs).
Seq. Id.: 8 Genbank/EMBL/DDBJ Accession No. NP 005903, from the National Center for Biotechnology Information—the amino acid sequence of the human MC 4-Receptor. (which has 1-332 amino acid residues).
Seq. Id.: 9 Genbank/EMBL/DDBJ Accession No. CAC01293, from the National Center for Biotechnology Information—the amino acid sequence of the human Agouti Signalling Protein (“ASP”) [Sus scrofa]. (which has 1-131 amino acid residues).
Seq. Id.: 10 Genbank/EMBL/DDBJ Accession No. AAK96256, from the National Center for Biotechnology Information—the amino acid sequence of the human Agouti Protein. (which has 1-132 amino acid residues).
Seq. Id.: 11 Genbank/EMBL/DDBJ Accession No. Q9TUK4, from the National Center for Biotechnology Information—the amino acid sequence of the Melanocyte stimulating hormone receptor (MSH-R). (which has 1-317 amino acid residues).
Seq. Id.: 12 Genbank/EMBL/DDBJ Accession No. Q01718, from the National Center for Biotechnology Information—the amino acid sequence of the Adrenocorticotropic hormone receptor (ACTH receptor) (ACTH-R). (which has 1-297 amino acid residues).
   Neuropeptide Y (NPY)

Neuropeptide Y (NPY) is a 97 amino acid peptide that has been found to be widely distributed throughout both the central and peripheral nervous systems. Several lines of evidence suggest that NPY plays a key role in the control of bodyweight. For example, central administration of NPY increases food intake and decreases thermogenesis in fed animals, while a reduction in endogenous NPY production leads to a decrease in food intake. Hypothalamic NPY peptide and mRNA levels are increased after fasting and in genetically obese mice generally. In fact, NPY is required for the maintenance of the obese phenotype of the leptin-deficient ob/ob mice. Conversely, leptin appears to decrease food intake and bodyweight in part by decreasing NPY synthesis and expression.

These data suggest that NPY may be a key modulator of bodyweight and that NPY receptor antagonists would be useful anti-obesity agents. NPY mediates its physiological effects via interaction with at least six distinct G protein-coupled receptors designated Y1 through Y6. The identity of the NPY receptor or receptors that control the regulation of food intake, energy expenditure and bodyweight by NPY is not completely clear, but recent evidence suggests that both the Y1 and Y5 receptors are involved. These pharmacological data implicate the Y1 and Y5 receptors as mediators of NPY-induced feeding. Development of additional non-peptide NPY receptor antagonists with superior pharmacokinetic properties relative to existing compounds may be effective against weight gain.
Seq. Id.: 13 Genbank/EMBL/DDBJ Accession No. NP000896, from the National Center for Biotechnology Information—the amino acid sequence of human Neuropeptide Y. (which has 1-97 amino acid residues).
Seq. Id.: 14 Genbank/EMBL/DDBJ Accession No. NP 00900, from the National Center for Biotechnology Information—the amino acid sequence of the human Neuropeptide Y Receptor Y1. (which has 1-384 amino acid residues).
Seq. Id.: 15 Genbank/EMBL/DDBJ Accession No. Q15761, from the National Center for Biotechnology Information—the amino acid sequence of the human Neuropeptide Y Receptor Y5. (which has 1-455 amino acid residues).
   Galanin

In humans, Galanin is a 30 amino acid non-amidated peptide. In other mammalian species, it is a 29 amino acid C-terminally amidated peptide. Originally isolated from porcine upper intestine, it has now been shown to be widely distributed in the brain and in peripheral tissues of several species. Crawley et al., demonstrated that central administration of galanin increased food intake in fed rats. Conversely, reduction of central galanin levels by antisense oligonucleotide techniques or central administration of a peptide galanin receptor antagonist decreased food intake. It is also known that galanin peptides and receptors are involved in the physiological coupling of body weight, adiposity and reproductive function.

These data suggest that galanin receptor antagonists may be useful anti-obesity agents. Galanin mediates its physiological effects via interaction with at least three distinct G protein-coupled receptors designated GALR1, GALR2 and GALR3. Transgenically produced and administered dysfunctional versions of receptors GALR1, GALR2 and GALR3 would be useful anti-obesity molecules.
Seq. Id.: 16 Genbank/EMBL/DDBJ Accession No. CAA 01907, from the National Center for Biotechnology Information—the amino acid sequence of the human Galanin. (which has 1-123 amino acid residues).
Seq. Id.: 17 Genbank/EMBL/DDBJ Accession No. NP 001471, from the National Center for Biotechnology Information—the amino acid sequence of the human Galanin Receptor 1. (which has 1-349 amino acid residues).
Seq. Id.: 18 Genbank/EMBL/DDBJ Accession No. O43603, from the National Center for Biotechnology Information—the amino acid sequence of the human Galanin Receptor 2. (which has 1-387 amino acid residues).
Seq. Id.: 19 Genbank/EMBL/DDBJ Accession No. O60755, from the National Center for Biotechnology Information—the amino acid sequence of the human Galanin Receptor 3. (which has 1-368 amino acid residues).
   Orexins

The orexins, orexin A and orexin B, were discovered as the endogenous ligands for an orphan G protein-coupled receptor originally found in the Human Genome Sciences database. Orexin A (33 amino acids) and orexin B (28 amino acids) are products of the same gene and are formed by proteolytic processing of a larger precursor molecule. While the orphan G protein-coupled receptor identified as the orexin receptor may in fact be an endogenous receptor for these peptides, neither orexin A nor orexin B has a particularly high affinity for this receptor, thus leaving open the possibility that another orexin receptor exists. Central administration of either orexin A or orexin B results in increased food intake. Like MCH, orexin neurons are primarily localized in the lateral hypothalamus and project to higher brain centers.

In addition, lateral hypothalamic orexin neurons expressing leptin receptors, are contacted by NPY/AGRP projections from the arcuate nucleus and send projections to arcuate NPY neurons. Orexin mRNA is also up-regulated by fasting. These data suggest that orexins play a significant role in the complex neuronal processes that regulate body weight. There are two orexin receptors, dysfunctional receptors that could still bind orexins would act to decrease weight.
Seq. Id.: 20 Genbank/EMBL/DDBJ Accession No. P56717, from the National Center for Biotechnology Information—the amino acid sequence of the human Orexin A. (which has 1-33 amino acid residues).
Seq. Id.: 21 Genbank/EMBL/DDBJ Accession No. O43612, from the National Center for Biotechnology Information—the amino acid sequence of the human Orexin B in a larger fragment (which has 1-28 amino acid residues).
Seq. Id.: 22 Genbank/EMBL/DDBJ Accession No. NP001516, from the National Center for Biotechnology Information—the amino acid sequence of the human Orexin Receptor 1. (which has 1-425 amino acid residues).
Seq. Id.: 23 Genbank/EMBL/DDBJ Accession No. NP 001517, from the National Center for Biotechnology Information—the amino acid sequence of the human Orexin Receptor 2. (which has 1-444 amino acid residues).

To recombinantly produce a protein of interest a nucleic acid encoding a transgenic protein can be introduced into a host cell, e.g., a cell of a primary or immortalized cell line. The recombinant cells can be used to produce the transgenic protein, including a cell surface receptor that can be secreted from a mammary epithelial cell. A nucleic acid encoding a transgenic protein can be introduced into a host cell, e.g., by homologous recombination. In most cases, a nucleic acid encoding the transgenic protein of interest is incorporated into a recombinant expression vector.

The nucleotide sequence encoding a transgenic protein can be operatively linked to one or more regulatory sequences, selected on the basis of the host cells to be used for expression. The term “operably linked” means that the sequences encoding the transgenic protein compound are linked to the regulatory sequence(s) in a manner that allows for expression of the transgenic protein. The term “regulatory sequence” refers to promoters, enhancers and other expression control elements (e.g., polyadenylation signals). Such regulatory sequences are described, for example, in Goeddel; GENE EXPRESSION TECHNOLOGY: METHODS IN ENZYMOLOGY 185, Academic Press, San Diego, Calif. (1990), the contents of which are incorporated herein by reference.

Regulatory sequences include those that direct constitutive expression of a nucleotide sequence in many types of host cells, those that direct expression of the nucleotide sequence only in certain host cells (e.g., tissue-specific regulatory sequences) and those that direct expression in a regulatable manner (e.g., only in the presence of an inducing agent). It will be appreciated by those skilled in the art that the design of the expression vector may depend on such factors as the choice of the host cell to be transformed, the level of expression of transgenic protein desired, and the like. The transgenic protein expression vectors can be introduced into host cells to thereby produce transgenic proteins encoded by nucleic acids.

Recombinant expression vectors can be designed for expression of transgenic proteins in prokaryotic or eukaryotic cells. For example, transgenic proteins can be expressed in bacterial cells such as E. coli, insect cells (e.g., in the baculovirus expression system), yeast cells or mammalian cells. Some suitable host cells are discussed further in Goeddel, GENE EXPRESSION TECHNOLOGY: METHODS IN ENZYMOLOGY 185, Academic Press, San Diego, Calif. (1990). Examples of vectors for expression in yeast S. cerevisiae include pYepSec1 (Baldari et al., (1987) EMBO J. 6:229-234), pMFa (Kurjan and Herskowitz, (1982) Cell 3:933-943), pJRY88 (Schultz et al., (1987) Gene 54:113-123), and pYES2 (Invitrogen Corporation, San Diego, Calif.). Baculovirus vectors available for expression of transgenic proteins in cultured insect cells include: the pAc series (Smith et al., (1983) MOL. CELL. BIOL. 3:2156-2165) and the pVL series (Lucklow, V. A., and Summers, M. D., (1989) VIROLOGY 170:31-39).

Examples of mammalian expression vectors include pCDM8 (Seed et al., (1987) NATURE 3:840) and pMT2PC (Kaufman et al. (1987), EMBO J. 6:187-195). When used in mammalian cells, the expression vector's control functions are often provided by viral regulatory elements. For example, commonly used promoters are derived from polyoma, Adenovirus 2, cytomegalovirus and SV40.

In addition to the regulatory control sequences discussed above, the recombinant expression vector can contain additional nucleotide sequences. For example, the recombinant expression vector may encode a selectable marker gene to identify host cells that have incorporated the vector. Moreover, to facilitate secretion of the transgenic protein from a host cell, in particular mammalian host cells, the recombinant expression vector can encode a signal sequence operatively linked to sequences encoding the amino-terminus of the transgenic protein such that upon expression, the transgenic protein is synthesized with the signal sequence fused to its amino terminus. This signal sequence directs the transgenic protein into the secretory pathway of the cell and is then cleaved, allowing for release of the mature transgenic protein (i.e., the transgenic protein without the signal sequence) from the host cell. Use of a signal sequence to facilitate secretion of proteins or peptides from mammalian host cells is known in the art.

Vector DNA can be introduced into prokaryotic or eukaryotic cells via conventional transformation or transfection techniques. As used herein, the terms “transformation” and “transfection” refer to a variety of art-recognized techniques for introducing foreign nucleic acid (e.g., DNA) into a host cell, including calcium phosphate or calcium chloride co-precipitation, DEAE-dextran-mediated transfection, lipofection, electroporation, microinjection and viral-mediated transfection. Suitable methods for transforming or transfecting host cells can be found in Sambrook et al. (Molecular Cloning: A Laboratory Manual, 2nd Edition, Cold Spring Harbor Laboratory press (1989)), and other laboratory manuals.

TABLE 1
Obesity Related Drugs Including the Thiazolidinediones
Drug List

| | |
|---|---|
| Regular insulin | Glyburide |
| Isophane insulin suspension (NPH) | Glipizide |
| Lente insulin | Glimepiride |
| Ultralente insulin | Metformin |
| Tolbutamide | Rosiglitazone |
| Acetohexamide | Pioglitazone |
| Chlorpropamide | Glucagon |
| Tolazamide | Repaglinide |
| Nateglinide | Troglitazone |
| Acarbose | Miglitol |
| Diazoxide | Propranolol |
| Glucagon | |

Materials and Methods
Transgenic Goats & Cattle

The herds of pure- and mixed-breed scrapie-free Alpine, Saanen and Toggenburg dairy goats used as cell and cell line donors for this study were maintained under Good Agricultural Practice (GAP) guidelines. Similarly, cattle used should be maintained under Good Agricultural Practice (GAP) guidelines and be certified to originate from a scrapie and bovine encephalitis free herd.

Isolation of Caprine Fetal Somatic Cell Lines.

Primary caprine fetal fibroblast cell lines to be used as karyoplast donors were derived from 35- and 40-day fetuses. Fetuses were surgically removed and placed in equilibrated phosphate-buffered saline (PBS, Ca⁺⁺/Mg⁺⁺-free). Single cell suspensions were prepared by mincing fetal tissue exposed to 0.025% trypsin, 0.5 mM EDTA at 38° C. for 10 minutes. Cells were washed with fetal cell medium [equilibrated Medium-199 (M199, Gibco) with 10% fetal bovine serum (FBS) supplemented with nucleosides, 0.1 mM 2-mercaptoethanol, 2 mM L-glutamine and 1% penicillin/streptomycin (10,000 I.U. each/ml)], and were cultured in 25 cm² flasks. A confluent monolayer of primary fetal cells was harvested by trypsinization after 4 days of incubation and then maintained in culture or cryopreserved.

Preparation of Donor Cells for Embryo Reconstruction.

Transfected fetal somatic cells were seeded in 4-well plates with fetal cell medium and maintained in culture (5% CO₂, 39° C.). After 48 hours, the medium was replaced with fresh low serum (0.5% FBS) fetal cell medium. The culture medium was replaced with low serum fetal cell medium every 48 to 72 hours over the next 2-7 days following low serum medium, somatic cells (to be used as karyoplast donors) were harvested by trypsinization. The cells were re-suspended in equilibrated M199 with 10% FBS supplemented with 2 mM L-glutamine, 1% penicillin/streptomycin (10,000 I. U. each/ml) for at least 6 hours prior to transgenic to the enucleated oocytes. The current experiments for the generation of desirable transgenic animals are preferably carried out with goat cells or mouse cells for the generation or goats or mice respectively but, according to the current invention, could be carried out with any mammalian cell line desired.

Oocyte Collection.

Oocyte donor does were synchronized and super ovulated as previously described (Ongeri, et al., 2001), and were mated to vasectomized males over a 48-hour interval. After collection, oocytes were cultured in equilibrated M199 with 10% FBS supplemented with 2 mM L-glutamine and 1% penicillin/streptomycin (10,000 I.U. each/ml).

Cytoplast Preparation and Enucleation.

All oocytes were treated with cytochalasin-B (Sigma, 5 μg/ml in SOF with 10% FBS) 15 to 30 minutes prior to enucleation. Metaphase-II stage oocytes were enucleated with a 25 to 30 μm glass pipette by aspirating the first polar body and adjacent cytoplasm surrounding the polar body (˜30% of the cytoplasm) to remove the metaphase plate. After enucleation, all oocytes were immediately reconstructed.

Nuclear Transfer and Reconstruction

Donor cell injection was conducted in the same medium used for oocyte enucleation. One donor cell was placed between the zona pellucida and the ooplasmic membrane using a glass pipet. The cell-oocyte couplets were incubated in SOF for 30 to 60 minutes before electrotransgenic and activation procedures. Reconstructed oocytes were equilibrated in transgenic buffer (300 mM mannitol, 0.05 mM CaCl₂, 0.1 mM MgSO₄, 1 mM K₂HPO₄, 0.1 mM glutathione, 0.1 mg/ml BSA) for 2 minutes. Electrotransgenic and activation were conducted at room temperature, in a transgenic chamber with 2 stainless steel electrodes fashioned into a “transgenic slide” (500 μm gap; BTX-Genetronics, San Diego, Calif.) filled with transgenic medium.

Transgenic was performed using a transgenic slide. The transgenic slide was placed inside a transgenic dish, and the dish was flooded with a sufficient amount of transgenic buffer to cover the electrodes of the transgenic slide. Couplets were removed from the culture incubator and washed through transgenic buffer. Using a stereomicroscope, couplets were placed equidistant between the electrodes, with the karyoplast/cytoplast junction parallel to the electrodes. It should be noted that the voltage range applied to the couplets to promote activation and transgenic can be from 1.0 kV/cm to 10.0 kV/cm. Preferably however, the initial single simultaneous transgenic and activation electrical pulse has a voltage range of 2.0 to 3.0 kV/cm, most preferably at 2.5 kV/cm, preferably for at least 20 μsec duration. This is applied to the cell couplet using a BTX ECM 2001 Electrocell Manipulator. The duration of the micropulse can vary from 10 to 80 μsec. After the process the treated couplet is typically transferred to a drop of fresh transgenic buffer. Transgenic treated couplets were washed through equilibrated SOF/FBS, then transferred to equilibrated SOF/FBS with or without cytochalasin-B. If cytocholasin-B is used its concentration can vary from 1 to 15 μg/ml, most preferably at 5 μg/ml. The couplets were incubated at 37-39° C. in a humidified gas chamber containing approximately 5% CO₂ in air. It should be noted that mannitol may be used in the place of cytocholasin-B throughout any of the protocols provided in the current disclosure (HEPES-buffered mannitol (0.3 mm) based medium with Ca⁺² and BSA).

Nuclear Transfer Embryo Culture and Transfer to Recipients.

Significant advances in nuclear transfer have occurred since the initial report of success in the sheep utilizing somatic cells (Wilmut et al., 1997). Many other species have since been cloned from somatic cells (Baguisi et al., 1999 and Cibelli et al., 1998) with varying degrees of success. Numerous other fetal and adult somatic tissue types (Zou et al., 2001 and Wells et al., 1999), as well as embryonic (Meng et al., 1997), have also been reported. The stage of cell cycle that the karyoplast is in at time of reconstruction has also been documented as critical in different laboratories methodologies (Kasinathan et al., BIOL. REPROD. 2001; Yong et al., 1998; and Kasinathan et al., NATURE BIOTECH. 2001).

All nuclear transfer embryos of the current invention were cultured in 50 μl droplets of SOF with 10% FBS overlaid with mineral oil. Embryo cultures were maintained in a humidified 39° C. incubator with 5% CO₂ for 48 hours before transfer of the embryos to recipient does. Recipient embryo transfer was performed as previously described (Baguisi et al., 1999).

Paramount to the success of any nuclear transfer program is having adequate transgenic of the karyoplast with the enucleated cytoplast. Equally important however is for that reconstructed embryo (karyoplast and cytoplast) to behave as a normal embryo and cleave and develop into a viable fetus and ultimately a live offspring. Results from this lab detailed above show that both transgenic and cleavage either separately or in combination have the ability to predict in a statistically significant fashion which cell lines are favorable to nuclear transfer procedures. While alone each parameter can aid in pre-selecting which cell line to utilize, in combination the outcome for selection of a cell line is strengthened.

Pregnancy and Perinatal Care.

For goats, pregnancy was determined by ultrasonography starting on day 25 after the first day of standing estrus. Does were evaluated weekly until day 75 of gestation, and once a month thereafter to assess fetal viability. For the pregnancy that continued beyond 152 days, parturition was induced with 5 mg of PGF2μ (Lutalyse, Upjohn). Parturition occurred within 24 hours after treatment. Kids were removed from the dam immediately after birth, and received heat-treated colostrum within 1 hour after delivery. Time frames appropriate for other ungulates with regard to pregnancy and perinatal care (e.g., bovines) are known in the art.

Cloned Animals.

The present invention also includes a method of cloning a genetically engineered or transgenic mammal, by which a desired gene is inserted, removed or modified in the differentiated mammalian cell or cell nucleus prior to insertion of the differentiated mammalian cell or cell nucleus into the enucleated oocyte.

Also provided by the present invention are mammals obtained according to the above method, and the offspring of those mammals. The present invention is preferably used for cloning caprines or bovines but could be used with any mammalian species. The present invention further provides for the use of nuclear transfer fetuses and nuclear transfer and chimeric offspring in the area of cell, tissue and organ transplantation.

Suitable mammalian sources for oocytes include goats, sheep, cows, pigs, rabbits, guinea pigs, mice, hamsters, rats, primates, etc. Preferably, the oocytes will be obtained from ungulates, and most preferably goats or cattle. Methods for isolation of oocytes are well known in the art. Essentially, this will comprise isolating oocytes from the ovaries or reproductive tract of a mammal, e.g., a goat. A readily available source of ungulate oocytes is from hormonally induced female animals.

For the successful use of techniques such as genetic engineering, nuclear transfer and cloning, oocytes may preferably be matured in vivo before these cells may be used as recipient cells for nuclear transfer, and before they can be fertilized by the sperm cell to develop into an embryo. Metaphase II stage oocytes, which have been matured in vivo, have been successfully used in nuclear transfer techniques. Essentially, mature metaphase II oocytes are collected surgically from either non-super ovulated or super ovulated animals several hours past the onset of estrus or past the injection of human chorionic gonadotropin (hCG) or similar hormone.

Moreover, it should be noted that the ability to modify animal genomes through transgenic technology offers new alternatives for the manufacture of recombinant proteins. The production of human recombinant pharmaceuticals in the milk of transgenic farm animals solves many of the problems associated with microbial bioreactors (e.g., lack of post-translational modifications, improper protein folding, high purification costs) or animal cell bioreactors (e.g., high capital costs, expensive culture media, low yields). The current invention enables the use of transgenic production of biopharmaceuticals, transgenic proteins, plasma proteins, and other molecules of interest in the milk or other bodily fluid (i.e., urine or blood) of transgenic animals homozygous for a desired gene.

According to an embodiment of the current invention when multiple or successive rounds of transgenic selection are utilized to generate a cell or cell line homozygous for more than one trait such a cell or cell line can be treated with compositions to lengthen the number of passes a given cell line can withstand in in vitro culture. Telomerase would be among such compounds that could be so utilized.

The use of living organisms as the production process means that all of the material produced will be chemically identical to the natural product. In terms of basic amino acid structures this means that only L-optical isomers, having the natural configuration, will be present in the product. Also the number of wrong sequences will be negligible because of the high fidelity of biological synthesis compared to chemical routes, in which the relative inefficiency of coupling reactions will always produce failed sequences. The absence of side reactions is also an important consideration with further modification reactions such as carboxy-terminal amidation. Again, the enzymes operating in vivo give a high degree of fidelity and stereospecificity which cannot be matched by chemical methods. Finally the production of a transgenic protein of interest in a biological fluid means that low-level contaminants remaining in the final product are likely to be far less toxic than those originating from a chemical reactor.

As previously mentioned, expression levels of three grams per liter of ovine milk are well within the reach of existing transgenic animal technology. Such levels should also be achievable for the recombinant proteins contemplated by the current invention.

In the practice of the present invention, obesity related transgenic proteins are produced in the milk of transgenic animals. The human recombinant protein of interest coding sequences can be obtained by screening libraries of genomic material or reverse-translated messenger RNA derived from the animal of choice (such as cattle or mice), or through appropriate sequence databases such as NCBI, genbank, etc. These sequences along with the desired polypeptide sequence of the transgenic partner protein are then cloned into an appropriate plasmid vector and amplified in a suitable host organism, usually E. coli. The DNA sequence encoding the peptide of choice can then be constructed, for example, by polymerase chain reaction amplification of a mixture of overlapping annealed oligonucleotides.

After amplification of the vector, the DNA construct would be excised with the appropriate 5′ and 3′ control sequences, purified away from the remains of the vector and used to produce transgenic animals that have integrated into their genome the desired obesity related transgenic protein. Conversely, with some vectors, such as yeast artificial chromosomes (YACs), it is not necessary to remove the assembled construct from the vector; in such cases the amplified vector may be used directly to make transgenic animals. In this case obesity related refers to the presence of a first polypeptide encoded by enough of a protein sequence nucleic acid sequence to retain its biological activity, this first polypeptide is then joined to a the coding sequence for a second polypeptide also containing enough of a polypeptide sequence of a protein to retain its physiological activity. The coding sequence being operatively linked to a control sequence which enables the coding sequence to be expressed in the milk of a transgenic non-human placental mammal.

A DNA sequence which is suitable for directing production to the milk of transgenic animals carries a 5′-promoter region derived from a naturally-derived milk protein and is consequently under the control of hormonal and tissue-specific factors. Such a promoter should therefore be most active in lactating mammary tissue. According to the current invention the promoter so utilized can be followed by a DNA sequence directing the production of a protein leader sequence which would direct the secretion of the transgenic protein across the mammary epithelium into the milk. At the other end of the transgenic protein construct a suitable 3′-sequence, preferably also derived from a naturally secreted milk protein, and may be added to improve stability of mRNA. An example of suitable control sequences for the production of proteins in the milk of transgenic animals are those from the caprine beta casein promoter.

The production of transgenic animals can now be performed using a variety of methods. The method preferred by the current invention is nuclear transfer.

Milk Specific Promoters.

The transcriptional promoters useful in practicing the present invention are those promoters that are preferentially activated in mammary epithelial cells, including promoters that control the genes encoding milk proteins such as caseins, beta-lacto globulin (Clark et al., (1989) BIO/TECHNOLOGY 7: 487-492), whey acid protein (Gorton et al. (1987) BIO/TECHNOLOGY 5: 1183-1187), and lactalbumin (Soulier et al., (1992) FEBS LETTS. 297: 13). Casein promoters may be derived from the alpha, beta, gamma or kappa casein genes of any mammalian species; a preferred promoter is derived from the goat beta casein gene (DiTullio, (1992) BIO/TECHNOLOGY 10:74-77). The milk-specific protein promoter or the promoters that are specifically activated in mammary tissue may be derived from either cDNA or genomic sequences. Preferably, they are genomic in origin.

DNA sequence information is available for all of the mammary gland specific genes listed above, in at least one, and often several organisms. See, e.g., Richards et al., J. BIOL. CHEM. 256, 526-532 (1981) α-lactalbumin rat); Campbell et al., NUCLEIC ACIDS RES. 12, 8685-8697 (1984) (rat WAP); Jones et al., J. BIOL. CHEM. 260, 7042-7050 (1985) (rat β-casein); Yu-Lee & Rosen, J. BIOL. CHEM. 258, 10794-10804 (1983) (rat γ-casein); Hall, BIOCHEM. J. 242, 735-742 (1987) (α-lactalbumin human); Stewart, NUCLEIC ACIDS RES. 12, 389 (1984) (bovine αs1 and κ casein cDNAs); Gorodetsky et al., GENE 66, 87-96 (1988) (bovine β casein); Alexander et al., EUR. J. BIOCHEM. 178, 395-401 (1988) (bovine κ casein); Brignon et al., FEBS LETT. 188, 48-55 (1977) (bovine αS2 casein); Jamieson et al., GENE 61, 85-90 (1987), Ivanov et al., BIOL. CHEM. Hoppe-Seyler 369, 425-429 (1988), Alexander et al., NUCLEIC ACIDS RES. 17, 6739 (1989) (bovine β lactoglobulin); Vilotte et al., BIOCHIMIE 69, 609-620 (1987) (bovine α-lactalbumin). The structure and function of the various milk protein genes are reviewed by Mercier & Vilotte, J. DAIRY SCI. 76, 3079-3098 (1993) (incorporated by reference in its entirety for all purposes). To the extent that additional sequence data might be required, sequences flanking the regions already obtained could be readily cloned using the existing sequences as probes. Mammary-gland specific regulatory sequences from different organisms are likewise obtained by screening libraries from such organisms using known cognate nucleotide sequences, or antibodies to cognate proteins as probes.

Signal Sequences.

Among the signal sequences that are useful in accordance with this invention are milk-specific signal sequences or other signal sequences which result in the secretion of eukaryotic or prokaryotic proteins. Preferably, the signal sequence is selected from milk-specific signal sequences, i.e., it is from a gene which encodes a product secreted into milk. Most preferably, the milk-specific signal sequence is related to the milk-specific promoter used in the expression system of this invention. The size of the signal sequence is not critical for this invention. All that is required is that the sequence be of a sufficient size to effect secretion of the desired recombinant protein, e.g., in the mammary tissue. For example, signal sequences from genes coding for caseins, e.g., alpha, beta, gamma or kappa caseins, beta lactoglobulin, whey acid protein, and lactalbumin are useful in the present invention. The preferred signal sequence is the goat β-casein signal sequence.

Signal sequences from other secreted proteins, e.g., proteins secreted by liver cells, kidney cell, or pancreatic cells can also be used.

Amino-Terminal Regions of Secreted Proteins.

The efficacy with which a non-secreted protein is secreted can be enhanced by inclusion in the protein to be secreted all or part of the coding sequence of a protein which is normally secreted. Preferably the entire sequence of the protein which is normally secreted is not included in the sequence of the protein but rather only a portion of the amino terminal end of the protein which is normally secreted. For example, a protein which is not normally secreted is fused (usually at its amino terminal end) to an amino terminal portion of a protein which is normally secreted.

Preferably, the protein which is normally secreted is a protein which is normally secreted in milk. Such proteins include proteins secreted by mammary epithelial cells, milk proteins such as caseins, beta lacto globulin, whey acid protein, and lactalbumin. Casein proteins include alpha, beta, gamma or kappa casein genes of any mammalian species. A preferred protein is beta casein, e.g., a goat beta casein. The sequences which encode the secreted protein can be derived from either cDNA or genomic sequences. Preferably, they are genomic in origin, and include one or more introns.

DNA Constructs.

The expression system or construct, described herein, can also include a 3′ untranslated region downstream of the DNA sequence coding for the non-secreted protein. This region apparently stabilizes the RNA transcript of the expression system and thus increases the yield of desired protein from the expression system. Among the 3′ untranslated regions useful in the constructs of this invention are sequences that provide a poly A signal. Such sequences may be derived, e.g., from the SV40 small t antigen, the casein 3′ untranslated region or other 3′ untranslated sequences well known in the art. Preferably, the 3′ untranslated region is derived from a milk specific protein. The length of the 3′ untranslated region is not critical but the stabilizing effect of its poly A transcript appears important in stabilizing the RNA of the expression sequence.

Optionally, the expression system or construct includes a 5′ untranslated region between the promoter and the DNA sequence encoding the signal sequence. Such untranslated regions can be from the same control region from which promoter is taken or can be from a different gene, e.g., they may be derived from other synthetic, semi-synthetic or natural sources. Again their specific length is not critical, however, they appear to be useful in improving the level of expression.

The construct can also include about 10%, 20%, 30%, or more of the N-terminal coding region of the gene preferentially expressed in mammary epithelial cells. For example, the N-terminal coding region can correspond to the promoter used, e.g., a goat β-casein N-terminal coding region.

The above-described expression systems may be prepared using methods well known in the art. For example, various ligation techniques employing conventional linkers, restriction sites etc. may be used to good effect. Preferably, the expression systems of this invention are prepared as part of larger plasmids. Such preparation allows the cloning and selection of the correct constructions in an efficient manner as is well known in the art. Most preferably, the expression systems of this invention are located between convenient restriction sites on the plasmid so that they can be easily isolated from the remaining plasmid sequences for incorporation into the desired mammal.

Prior art methods often include making a construct and testing it for the ability to produce a product in cultured cells prior to placing the construct in a transgenic animal. Surprisingly, the inventors have found that such a protocol may not be of predictive value in determining if a normally non-secreted protein can be secreted, e.g., in the milk of a transgenic animal. Therefore, it may be desirable to test constructs directly in transgenic animals, e.g., transgenic mice, as some constructs which fail to be secreted in CHO cells are secreted into the milk of transgenic animals.

Sequence Production and Modification

The invention encompasses the use of the described nucleic acid sequences and the peptides expressed therefrom in various transgenic animals. The sequences of specific molecules can be manipulated to generate proteins that retain most of their tertiary structure but are physiologically non-functional.

PCR technology may also be utilized to isolate full length cDNA sequences. For example, RNA may be isolated, following standard procedures, from an appropriate cellular or tissue source (i.e., one known, or suspected, to express a target receptor gene, such as, for example from, skin, testis, or brain tissue). A reverse transcription (RT) reaction may be performed on the RNA using an oligonucleotide primer specific for the most 5′ end of the amplified fragment for the priming of first strand synthesis. The resulting RNA/DNA hybrid may then be “tailed” using a standard terminal transferase reaction, the hybrid may be digested with RNase H, and second strand synthesis may then be primed with a complementary primer. Thus, cDNA sequences upstream of the amplified fragment may easily be isolated. For a review of cloning strategies which may be used, see e.g., Sambrook et al., 1989.

A cDNA of a mutant target gene may be isolated, for example, by using PCR. In this case, the first cDNA strand may be synthesized by hybridizing an oligo-dT oligonucleotide to mRNA isolated from tissue known or suspected to be expressed in an individual putatively carrying a mutant target allele, and by extending the new strand with reverse transcriptase. The second strand of the cDNA is then synthesized using an oligonucleotide that hybridizes specifically to the 5′ end of the normal gene. Using these two primers, the product is then amplified via PCR, optionally cloned into a suitable vector, and subjected to DNA sequence analysis through methods well known to those of skill in the art. By comparing the DNA sequence of the mutant target allele to that of the normal target allele, the mutation(s) responsible for the loss or alteration of function of the mutant target gene product can be ascertained.

Alternatively, a genomic library can be constructed using DNA obtained from an individual suspected of or known to carry the mutant target allele, or a cDNA library can be constructed using RNA from a tissue known, or suspected, to express the mutant target allele. A normal target gene, or any suitable fragment thereof, can then be labeled and used as a probe to identify the corresponding mutant target allele in such libraries. Clones containing the mutant target gene sequences may then be purified and subjected to sequence analysis according to methods well known to those of skill in the art.

Additionally, an expression library can be constructed utilizing cDNA synthesized from, for example, RNA isolated from a tissue known, or suspected, to express a mutant target allele in an individual suspected of or known to carry such a mutant allele. In this manner, gene products made by the putatively mutant tissue may be expressed and screened using standard antibody screening techniques in conjunction with antibodies raised against the normal target product.

The invention also encompasses nucleotide sequences that encode mutant target receptor protein sequences, peptide fragments of the target receptor proteins, truncated target receptor proteins, and target receptor protein fusion proteins. These include, but are not limited to nucleotide sequences encoding mutant target receptor proteins described herein; polypeptides or peptides corresponding to one or more domains of the target receptor protein or portions of these domains; truncated target receptor protein in which one or more of the domains is purposefully deleted, or a truncated non-functional target receptor protein so as to generate a purposefully dysfunctional receptor protein.

Purposefully dysfunctional receptor proteins can be made and expressed in a transgenic system to provide a composition that can bind to physiological agents that would maintain obesity or work to increase weight gain. Nucleotides encoding fusion proteins may include, but are not limited to, full length target receptor protein sequences, truncated target receptor proteins, or nucleotides encoding peptide fragments of a target receptor protein fused to an unrelated protein or peptide that will facilitate expression in a transgenic mammal or other transgenic animal expression model, such as for example, a target receptor protein domain fused to an Ig Fc domain which increases the stability and half-life of the resulting fusion protein in the bloodstream such that retains its ability to ameliorate obesity or related pathologies.

The target receptor protein amino acid sequences of the invention include the amino acid sequences presented in the sequence listings herein as well as analogues and derivatives thereof. Further, corresponding target receptor protein homologues from other species are encompassed by the invention. The degenerate nature of the genetic code is well known, and, accordingly, each amino acid presented in the sequence listings, is generically representative of the well known nucleic acid “triplet” codon, or in many cases codons, that can encode the amino acid. As such, as contemplated herein, the amino acid sequences presented in the sequence listing, when taken together with the genetic code (see, pp 109, Table 4-1 of MOLECULAR CELL BIOLOGY, (1986), J. Darnell et al. eds., incorporated by reference) are generically representative of all the various permutations and combinations of nucleic acid sequences that can encode such amino acid sequences.

According to a preferred embodiment of the invention random mutations can be made to target gene DNA through the use of random mutagenesis techniques well known to those skilled in the art with the resulting mutant target receptor proteins tested for activity, site-directed mutations of the target receptor protein coding sequence can be engineered to generate mutant target receptor proteins with the same structure but with limited physiological function, e.g., alternate function, and/or with increased half-life. This can be accomplished using site-directed mutagenesis techniques well known to those skilled in the art.

One starting point for such activities is to align the disclosed human sequences with corresponding gene/protein sequences from, for example, other mammals in order to identify specific amino acid sequence motifs within the target gene that are conserved between different species. Changes to conserved sequences can be engineered to alter function, signal transduction capability, or both. Alternatively, where the alteration of function is desired, deletion or non-conservative alterations of the conserved regions can also be engineered.

Other mutations to the target protein coding sequence can be made to generate target proteins that are better suited for expression, scale-up, etc. in the host cells chosen. For example, cysteine residues can be deleted or substituted with another amino acid in order to eliminate disulfide bridges.

While the target proteins and peptides can be chemically synthesized, large sequences derived from a target protein and full length gene sequences can be advantageously produced by recombinant DNA technology using techniques well known in the art for expressing nucleic acid containing target protein gene sequences and/or nucleic acid coding sequences. Such methods can be used to construct expression vectors containing appropriate transcriptional and translational control signals. These methods include, for example, in vitro recombinant DNA techniques, synthetic techniques, and in vivo genetic recombination.

Transgenic Mammals.

Preferably, the DNA constructs of the invention are introduced into the germ-line of a mammal. For example, one or several copies of the construct may be incorporated into the genome of a mammalian embryo by standard transgenic techniques known in the art.

Any non-human mammal can be usefully employed in this invention. Mammals are defined herein as all animals, excluding humans, which have mammary glands and produce milk. Preferably, mammals that produce large volumes of milk and have long lactating periods are preferred. Preferred mammals are cows, sheep, goats, mice, oxen, camels and pigs. Of course, each of these mammals may not be as effective as the others with respect to any given expression sequence of this invention. For example, a particular milk-specific promoter or signal sequence may be more effective in one mammal than in others. However, one of skill in the art may easily make such choices by following the teachings of this invention.

In an exemplary embodiment of the current invention, a transgenic non-human animal is produced by introducing a transgene into the germline of the non-human animal. Transgenes can be introduced into embryonal target cells at various developmental stages. Different methods are used depending on the stage of development of the embryonal target cell. The specific line(s) of any animal used should, if possible, be selected for general good health, good embryo yields, good pronuclear visibility in the embryo, and good reproductive fitness.

The litters of transgenic mammals may be assayed after birth for the incorporation of the construct into the genome of the offspring. Preferably, this assay is accomplished by hybridizing a probe corresponding to the DNA sequence coding for the desired recombinant protein product or a segment thereof onto chromosomal material from the progeny. Those mammalian progeny found to contain at least one copy of the construct in their genome are grown to maturity. The female species of these progeny will produce the desired protein in or along with their milk. Alternatively, the transgenic mammals may be bred to produce other transgenic progeny useful in producing the desired proteins in their milk.

In accordance with the methods of the current invention for transgenic animals a transgenic primary cell line (from either caprine, bovine, ovine, porcine or any other non-human vertebrate origin) suitable for somatic cell nuclear transfer is created by transfection of the transgenic protein nucleic acid construct of interest (for example, a mammary gland-specific transgene(s) targeting expression of a transgenic protein to the mammary gland). The transgene construct can either contain a selection marker (such as neomycin, kanamycin, tetracycline, puromycin, zeocin, hygromycin or any other selectable marker) or be co-transfected with a cassette able to express the selection marker in cell culture.

Transgenic females may be tested for protein secretion into milk, using any of the assay techniques that are standard in the art (e.g., Western blots or enzymatic assays).

The invention provides expression vectors containing a nucleic acid sequence described herein, operably linked to at least one regulatory sequence. Many such vectors are commercially available, and other suitable vectors can be readily prepared by the skilled artisan. “Operably linked” or “operatively linked” is intended to mean that the nucleic acid molecule is linked to a regulatory sequence in a manner which allows expression of the nucleic acid sequence by a host organism. Regulatory sequences are art recognized and are selected to produce the encoded polypeptide or protein. Accordingly, the term “regulatory sequence” includes promoters, enhancers, and other expression control elements which are described in Goeddel, GENE EXPRESSION TECHNOLOGY: METHODS IN ENZYMOLOGY 185, (Academic Press, San Diego, Calif. (1990)). For example, the native regulatory sequences or regulatory sequences native to the transformed host cell can be employed.

It should be understood that the design of the expression vector may depend on such factors as the choice of the host cell to be transformed and/or the type of protein desired to be expressed. For instance, the polypeptides of the present invention can be produced by ligating the cloned gene, or a portion thereof, into a vector suitable for expression in either prokaryotic cells, eukaryotic cells or both. (A LABORATORY MANUAL, 2nd Ed., ed. Sambrook et al. (Cold Spring Harbor Laboratory Press, 1989) Chapters 16 and 17)).

Following selection of colonies recombinant for the desired nucleic acid construct, cells are isolated and expanded, with aliquots frozen for long-term preservation according to procedures known in the field. The selected transgenic cell-lines can be characterized using standard molecular biology methods (PCR, Southern blotting, FISH). Cell lines carrying nucleic acid constructs of the obesity related transgenic protein of interest, of the appropriate copy number, generally with a single integration site (although the same technique could be used with multiple integration sites) can then be used as karyoplast donors in a somatic cell nuclear transfer protocol known in the art. Following nuclear transfer, and embryo transfer to a recipient animal, and gestation, live transgenic offspring are obtained.

Typically this transgenic offspring carries only one transgene integration on a specific chromosome, the other homologous chromosome not carrying an integration in the same site. Hence the transgenic offspring is heterozygous for the transgene, maintaining the current need for at least two successive breeding cycles to generate a homozygous transgenic animal.

Genetic Marker

Often only a small fraction of mammalian cells integrate the foreign DNA into their genome. In order to identify and select these integrants, a gene that encodes a selectable marker (e.g., resistance to antibiotics) can be introduced into the host cells along with the gene encoding the transgenic protein. Preferred selectable markers include those that confer resistance to drugs, such as G418, hygromycin and methotrexate. Nucleic acid encoding a selectable marker can be introduced into a host cell on the same vector as that encoding the transgenic protein or can be introduced on a separate vector. Cells stably transfected with the introduced nucleic acid can be identified by drug selection (e.g., cells that have incorporated the selectable marker gene will survive, while the other cells die).

Animal Promoters

Useful promoters for the expression of obesity related in mammary tissue include promoters that naturally drive the expression of mammary-specific polypeptides, such as milk proteins, although any promoter that permits secretion of obesity related into milk can be used. These include, e.g., promoters that naturally direct expression of whey acidic protein (WAP), alpha S1-casein, alpha S2-casein, beta-casein, kappa-casein, beta-lactoglobulin, alpha-lactalbumin (see, e.g., Drohan et al., U.S. Pat. No. 5,589,604; Meade et al., U.S. Pat. No. 4,873,316; and Karatzas et al., U.S. Pat. No. 5,780,009), and others described in U.S. Pat. No. 5,750,172. Whey acidic protein (WAP; Genbank Accession No. X01153), the major whey protein in rodents, is expressed at high levels exclusively in the mammary gland during late pregnancy and lactation (Hobbs et al., J. BIOL. CHEM. 257:3598-3605, 1982). For additional information on desired mammary gland-specific promoters, see, e.g., Richards et al., J. BIOL. CHEM. 256:526-532, 1981 (α-lactalbumin rat); Campbell et al., NUCLEIC ACIDS RES. 12:8685-8697, 1984 (rat WAP); Jones et al., J. BIOL. CHEM. 260:7042-7050, 1985 (rat β-casein); Yu-Lee & Rosen, J. BIOL. CHEM. 258:10794-10804, 1983 (rat γ-casein); Hall, BIOCHEM. J. 242:735-742, 1987 (human α-lactalbumin); Stewart, NUCLEIC ACIDS RES. 12:3895-3907, 1984 (bovine α-s1 and κ-casein cDNAs); Gorodetsky et al., GENE 66:87-96, 1988 (bovine β-casein); Alexander et al., EUR. J. BIOCHEM. 178:395-401, 1988 (bovine κ-casein); Brignon et al., FEBS LETT. 188:48-55, 1977 (bovine α-S2 casein); Jamieson et al., GENE 61:85-90, 1987, Ivanov et al., BIOL. CHEM. Hoppe-Seyler 369:425-429, 1988, and Alexander et al., NUCLEIC ACIDS RES. 17:6739, 1989 (bovine β-lactoglobulin); and Vilotte et al., BIOCHIMIE 69:609-620, 1987 (bovine α-lactalbumin). The structure and function of the various milk protein genes are reviewed by Mercier & Vilotte, J. DAIRY SCI. 76:3079-3098, 1993.

If additional flanking sequences are useful in optimizing expression, such sequences can be cloned using the existing sequences as probes. Mammary-gland specific regulatory sequences from different organisms can be obtained by screening libraries from such organisms using known cognate nucleotide sequences, or antibodies to cognate proteins as probes.

Useful signal sequences for expression and secretion of obesity related into milk are milk-specific signal sequences. Desirably, the signal sequence is selected from milk-specific signal sequences, i.e., from a gene which encodes a product secreted into milk. Most desirably, the milk-specific signal sequence is related to a milk-specific promoter described above. The size of the signal sequence is not critical for this invention. All that is required is that the sequence be of a sufficient size to effect secretion of a target transgenic protein of use in the treatment of obesity, e.g., in the mammary tissue. For example, signal sequences from genes coding for caseins, e.g., alpha, beta, gamma, or kappa caseins, beta lactoglobulin, whey acidic protein, and lactalbumin are useful in the present invention. Signal sequences from other secreted proteins, e.g., proteins secreted by liver cells, kidney cell, or pancreatic cells can also be used.

Useful promoters for the expression of a recombinant polypeptide transgene in urinary tissue are the uroplakin and uromodulin promoters (Kerr et al., NAT. BIOTECHNOL. 16:75-79, 1998; Zbikowska, et al., BIOCHEM. J. 365:7-11, 2002; and Zbikowski et al., TRANSGENIC RES. 11:425-435, 2002), although any promoter that permits secretion of the transgene product into urine may be used.

A useful promoter for the expression and secretion of obesity related into blood by blood-producing or serum-producing cells (e.g., liver epithelial cells) is the albumin promoter (see, e.g., Shen et al., DNA 8:101-108, 1989; Tan et al., DEV. BIOL. 146:24-37, 1991; McGrane et al., TIBS 17:40-44, 1992; Jones et al., J. BIOL. CHEM. 265:14684-14690, 1990; and Shimada et al., FEBS LETTERS 279:198-200, 1991), although any promoter that permits secretion of the transgene product into blood may be used. The native alpha-fetoprotein promoter can also be used (see, e.g., Genbank Accession Nos.: AB053574; AB053573; AB053572; AB053571; AB053570; and AB053569). Useful promoters for the expression of obesity related in semen are described in U.S. Pat. No. 6,201,167. Useful avian-specific promoters are the ovalbumin promoter and the apo-B promoter.

Another three grams is produced in the liver (serum lipoproteins) and deposited in the egg yolk. In addition, since birds do not typically recognize mammalian proteins immunologically because of their evolutionary distance from mammals, the expression of obesity related in birds is less likely to have any deleterious effect on the viability and health of the bird.

Other promoters that are useful in the methods of the invention include inducible promoters. Generally, recombinant proteins are expressed in a constitutive manner in most eukaryotic expression systems. The addition of inducible promoters or enhancer elements provides temporal or spatial control over expression of the transgenic proteins of interest, and provides an alternative mechanism of expression. Inducible promoters include heat shock protein, metallothionien, and MMTV-LTR, while inducible enhancer elements include those for ecdysone, muristerone A, and tetracycline/doxycycline.

Nucleic Acid Vectors

In certain embodiments the invention concerns vectors, or recombinant expression vectors, comprising any of the nucleic acid molecules described herein. Vectors are used herein either to amplify DNA or RNA encoding transgenic proteins and/or to express DNA which encodes SSTR-transgenic proteins. Vectors include, but are not limited to, plasmids, phages, cosmids, episomes, viral particles or viruses, and integratable DNA fragments (i.e., fragments integratable into the host genome by homologous recombination). Viral particles include, but are not limited to, adenoviruses, baculoviruses, parvoviruses, herpesviruses, poxviruses, adeno-associated viruses, vaccinia viruses, retroviruses, microparticles and naked DNA. In various embodiments, expression may be targeted to a particular cell type or cell population by a targeting ligand. Expression vectors include, but are not limited to, pcDNA3 (Invitrogen) and pSVL (Pharmacia Biotech). Other expression vectors include, but are not limited to, pSPORT™ vectors, pGEM™ vectors (Promega), Bluescript™ vectors (Stratagene), pSE420™ (Invitrogen), and pYES2™ (Invitrogen).

Expression constructs may comprise a transgenic protein encoding polynucleotides operatively linked to an endogenous or exogenous expression control DNA sequence and a transcription terminator. Because of limited space for nucleic acid insertion in many vectors it may be desirable to insert smaller reporters or reporter transgenic constructs. For example, deletion of all or part of the somatosatin receptor carboxy terminus may be used. Expression control DNA sequences include promoters, enhancers, operators, and regulatory element binding sites generally, and are typically selected based on the expression systems in which the expression construct is to be utilized.

Promoter and enhancer sequences are generally selected for the ability to increase gene expression, while operator sequences are generally selected for the ability to regulate gene expression. Expression constructs of the invention may also include sequences encoding one or more selectable markers that permit identification of host cells bearing the construct. Expression constructs may also include sequences that facilitate homologous recombination in a host cell. In various embodiments constructs may also include sequences necessary for replication in a host cell.

Various exemplary tissue-specific promoters are listed herein (Pearse and Takor, 1979; Nylen and Becker, 1995). Although not a complete list, these promoters are exemplary of the types of promoters and enhancers that may be used in certain embodiments of the invention. Additional promoters, useful in the present invention, will be readily known to those of skill in the art.

Inducible promoters include but are not limited to MT II, MMTV (mouse mammary tumor virus), c-jun, Collagenase, Stromelysin, Murine MX Gene, GRP78 Gene, α-2-Macroglobulin, Vimentin, MHC Class I Gene H-2 kB, HSP70, Proliferin, Tumor Necrosis Factor and Thyroid Stimulating Hormone-α. Cell or tissue specific expression can be achieved by using cell-specific enhancers and/or promoters. (See generally, Huber et al., ADV. DRUG DELIVERY REVIEWS 17:279-292, 1995).

Expression constructs may be utilized for production of an encoded protein, but may also be utilized simply to amplify an SSTR-transgenic protein encoding polynucleotide sequence. In some embodiments, the vector is an expression vector wherein the polynucleotide is operatively linked to a polynucleotide comprising an expression control sequence. In certain embodiments autonomously replicating recombinant expression constructs such as plasmid and viral DNA vectors incorporating polynucleotides. Expression vectors may be replicable DNA constructs in which a DNA sequence encoding SSTR-transgenic protein is operably linked or connected to suitable control sequences capable of effecting the expression of an SSTR-transgenic protein in a suitable host. DNA regions are operably linked or connected when they are functionally related to each other. For example, a promoter is operably linked or connected to a coding sequence if it controls the transcription of the sequence. Amplification vectors do not require expression control domains, but rather need only the ability to replicate in a host, usually conferred by an origin of replication, and a selection gene to facilitate recognition of transformants. The need for control sequences in the expression vector will vary depending upon the host selected and the transformation method chosen. Generally, control sequences include a transcriptional promoter, an optional operator sequence to control transcription, a sequence encoding suitable mRNA ribosomal binding and sequences that controls the termination of transcription and translation.

In various embodiments vectors may contain a promoter that is recognized by the host organism. The promoter sequences may be prokaryotic, eukaryotic, synthetic or viral. Examples of suitable prokaryotic sequences include the promoters of bacteriophage lambda (THE BACTERIOPHAGE LAMBDA, Hershey, A. D., Ed., Cold Spring Harbor Press, Cold Spring Harbor, N.Y. (1973); LAMBDA II, Hendrix, R. W., Ed., Cold Spring Harbor Press, Cold Spring Harbor, N.Y. (1980); and, Benoist et al., The trp, recA, heat shock, and lacZ promoters of E. coli and the SV40 early promote, NATURE, 290:304-310, (1981). Additional promoters include, but are not limited to, mouse mammary tumor virus, long terminal repeat of human immunodeficiency virus, maloney virus, cytomegalovirus immediate early promoter, Epstein Barr virus, Rous sarcoma virus, human actin, human myosin, human hemoglobin, human muscle creatine, and human metalothionein.

Additional regulatory sequences may also be included in vectors. Examples of suitable regulatory sequences are represented by the Shine-Dalgarno of the replicase gene of the phage MS-2 and of the gene cII of bacteriophage lambda. The Shine-Dalgarno sequence may be directly followed by DNA encoding SSTR-transgenic protein and result in the expression of the mature SSTR-transgenic protein.

Moreover, suitable expression vectors can include an appropriate marker that allows the screening of the transformed host cells. The transformation of the selected host is carried out using any one of the various techniques well known to the expert in the art and described in Sambrook et al., supra.

An origin of replication may also be provided either by construction of the vector to include an exogenous origin or may be provided by the host cell chromosomal replication mechanism. If the vector is integrated into the host cell chromosome, the latter may be sufficient. Alternatively, rather than using vectors which contain viral origins of replication, one skilled in the art can transform mammalian cells by the method of co-transformation with a selectable marker and SSTR-transgenic protein encoding DNA. An example of a suitable marker is dihydrofolate reductase or thymidine kinase (see, U.S. Pat. No. 4,399,216).

Nucleotide sequences encoding reporter protein transgenics, such as SSTR2-transgenic proteins, may be recombined with vector DNA in accordance with conventional techniques, including blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases. Techniques for such manipulation are disclosed by Sambrook et al., supra and are well known in the art. Methods for construction of mammalian expression vectors are disclosed in, for example, Okayama et al., MOL. CELL. BIOL., 3:280, (1983); Cosman et al., MOL. IMMUNOL., 23:935, (1986); and, Cosman et al., NATURE, 312: 768, (1984).

The transgene construct preferably includes a leader sequence downstream from the promoter. The leader sequence is a nucleic acid sequence that encodes a protein secretory signal, and, when operably linked to a downstream nucleic acid molecule encoding the obesity related transgenic protein of the invention, and directs secretion of the desired protein. The leader sequence may be obtained from the same gene as the promoter used to direct transcription of the nucleic acid molecule encoding obesity related (for example, a gene that encodes a milk-specific protein). Alternatively, a leader sequence encoding the native human obesity related protein secretory signal (amino acids 1-19 of Genbank Accession No. V01514) may be employed.

Therapeutic Uses.

The combination herein is preferably employed for in vitro use in treating these tissue cultures. The combination, however, is also be effective for in vivo applications. Depending on the intended mode of administration in vivo the compositions used may be in the dosage form of solid, semi-solid or liquid such as, e.g., tablets, pills, powders, capsules, gels, ointments, liquids, suspensions, or the like. Preferably the compositions are administered in unit dosage forms suitable for single administration of precise dosage amounts. The compositions may also include, depending on the formulation desired, pharmaceutically acceptable carriers or diluents, which are defined as aqueous-based vehicles commonly used to formulate pharmaceutical compositions for animal or human administration. The diluent is selected so as not to affect the biological activity of the human recombinant protein of interest. Examples of such diluents are distilled water, physiological saline, Ringer's solution, dextrose solution, and Hank's solution. The same diluents may be used to reconstitute lyophilized a human recombinant protein of interest. In addition, the pharmaceutical composition may also include other medicinal agents, pharmaceutical agents, carriers, adjuvants, nontoxic, non-therapeutic, non-immunogenic stabilizers, etc. Effective amounts of such diluent or carrier will be amounts which are effective to obtain a pharmaceutically acceptable formulation in terms of solubility of components, biological activity, etc.

The compositions herein may be administered to human patients via oral, parenteral or topical administrations and otherwise systemic forms for anti-melanoma and anti-breast cancer treatment.

Bacterial Expression.

Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector and, if desirable, to provide amplification within the host. Suitable prokaryotic hosts for transformation include E. coli, Bacillus subtilis, Salmonella typhimurium and various species within the genera Pseudomonas, Streptomyces, and Staphylococcus, although others may, also be employed as a matter of choice. In a preferred embodiment, the prokaryotic host is E. coli.

Bacterial vectors may be, for example, bacteriophage-, plasmid- or cosmid-based. These vectors can comprise a selectable marker and bacterial origin of replication derived from commercially available plasmids typically containing elements of the well known cloning vector pBR322 (ATCC 37017). Such commercial vectors include, for example, GEM 1 (Promega Biotec, Madison, Wis., USA), pBs, phagescript, PsiX174, pBluescript SK, pBs KS, pNH8a, pNH16a, pNH18a, pNH46a (Stratagene); pTrc99A, pKK223-3, pKK233-3, pKK232-8, pDR540, and pRIT5 (Pharmacia). A preferred vector according to the invention is THE Pt7I expression vector.

These “backbone” sections are combined with an appropriate promoter and the structural sequence to be expressed. Bacterial promoters include lac, T3, T7, lambda PR or PL, trp, and ara. T7 is a preferred bacterial promoter.

Following transformation of a suitable host strain and growth of the host strain to an appropriate cell density, the selected promoter is de-repressed/induced by appropriate means (e.g., temperature shift or chemical induction) and cells are cultured for an additional period. Cells are typically harvested by centrifugation, disrupted by physical or chemical means, and the resulting crude extract retained for further purification.

Eukaryotic Expression Vectors

Various mammalian cell culture systems can also be employed to express recombinant proteins. Examples of mammalian expression systems include selected mouse L cells, such as thymidine kinase-negative (TK) and adenine phosphoribosul transferase-negative (APRT) cells. Other examples include the COS-7 lines of monkey kidney fibroblasts, described by Gluzman, CELL 23:175 (1981), and other cell lines capable of expressing a compatible vector, for example, the C127, 3T3, CHO, HeLa and BHK cell lines. In particular, as regards yeasts, there may be mentioned yeasts of the genus Saccharomyces, Kluyveromyces, Pichia, Schwanniomyces, or Hansenula. Among the fungi capable of being used in the present invention, there may be mentioned more particularly Aspergillus ssp, or Trichoderma ssp.

Mammalian expression vectors will comprise an origin of replication, a suitable promoter and enhancer, and also any necessary ribosome binding sites, polyadenylation site, splice donor and acceptor sites, transcriptional termination sequences, and 5′ flanking non-transcribed sequences. DNA sequences derived from the SV40 viral genome, for example, SV40 origin, early promoter, enhancer, splice, and polyadenylation sites may be used to provide the required non-transcribed genetic elements.

Mammalian promoters include beta-casein, beta-lactoglobulin, whey acid promoter others include: HSV thymidine kinase, early and late SV40, LTRs from retrovirus, and mouse metallothionein-1. Exemplary mammalian vectors include pWLneo, pSV2cat, pOG44, pXT1, pSG (Stratagene) pSVK3, pBPV, pMSG, and pSVL (Pharmacia). In a preferred embodiment, the mammalian expression vector is pUCIG-MET. Selectable markers include CAT (chloramphenicol transferase).

The nucleotide sequences which can be used within the framework of the present invention can be prepared in various ways. Generally, they are obtained by assembling, in reading phase, the sequences encoding each of the functional parts of the polypeptide. The latter may be isolated by the techniques of persons skilled in the art, and for example directly from cellular messenger RNAs (mRNAs), or by recloning from a complementary DNA (cDNA) library, or alternatively they may be completely synthetic nucleotide sequences. It is understood, furthermore, that the nucleotide sequences may also be subsequently modified, for example by the techniques of genetic engineering, in order to obtain derivatives or variants of the said sequences.

Fluorescence In Situ Hybridization (FISH) Analysis.

Standard culture and preparation procedures are used to obtain metaphase and interphase nuclei from cultured cells derived from animals carrying the desirable transgene. Nuclei are deposited onto slides and were hybridized with a digoxigenin-labeled probe derived from a construct containing 8 kb of the genomic sequence for the obesity related protein of interest. Bound probe was amplified using a horseradish peroxidase-conjugated antibody and detected with tyramide-conjugated fluorescein isothiocyanate (FITC, green fluorochrome). Nuclei were counterstained with 4′, 6-diamidino-2-phenylindole (DAPI, blue dye). FISH images were obtained using MetaMorph software.

Therapeutic Compositions.

The proteins of the present invention can be formulated according to known methods to prepare pharmaceutically useful compositions, whereby the inventive molecules, or their functional derivatives, are combined in admixture with a pharmaceutically acceptable carrier vehicle. Suitable vehicles and their formulation, inclusive of other human proteins, e.g., human serum albumin, are described, for example, in order to form a pharmaceutically acceptable composition suitable for effective administration, such compositions will contain an effective amount of one or more of the proteins of the present invention, together with a suitable amount of carrier vehicle.

Pharmaceutical compositions for use in accordance with the present invention may be formulated in conventional manner using one or more physiologically acceptable carriers or excipients. Thus, the obesity related molecules and their physiologically acceptable salts and solvate may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the composition may take the form of tablets or lozenges formulated in conventional manner.

For administration by inhalation, the obesity related molecules for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethan-e, carbon dioxide or other suitable gas. In the case of a pressurized aerosol the dosage unit may be determined by providing a valve to deliver a metered amount. Capsules and cartridges of, e.g. gelatin for use in an inhaler or insufflator may be formulated containing a powder mix of the compound and a suitable powder base such as lactose or starch.

The obesity related transgenic proteins of the invention may be formulated for parenteral administration by injection, e.g., by bolus injection or continuous intransgenic. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the obesity related molecules may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Treatment Methods.

The inventive therapeutic methods according to the invention generally utilize the obesity related proteins identified above. The domains of the transgenic proteins share the ability to specifically target a specific tissue and/or augment an immune response to targeted tissue. A typical method, accordingly, involves binding a receptor of a targeted cell to the receptor-antagonizing domain of the transgenic protein and/or stimulating a T-cell dependent immune response.

Therapeutic methods involve administering to a subject in need of treatment a therapeutically effective amount of a transgenic protein. “Therapeutically effective” is employed here to denote the amount of transgenic proteins that are of sufficient quantity to inhibit or reverse a disease condition (e.g., reduce or inhibit cancer growth). Some methods contemplate combination therapy with known cancer medicaments or therapies, for example, chemotherapy (preferably using compounds of the sort listed above) or radiation. The patient may be a human or non-human animal. A patient typically will be in need of treatment when suffering from a cancer characterized by increased levels of receptors that promote cancer maintenance or proliferation.

Administration during in vivo treatment may be by any number of routes, including parenteral and oral, but preferably parenteral. Intracapsular, intravenous, intrathecal, and intraperitoneal routes of administration may be employed, generally intravenous is preferred. The skilled artisan will recognize that the route of administration will vary depending on the disorder to be treated.

Determining a therapeutically effective amount specifically will depend on such factors as toxicity and efficacy of the medicament. Toxicity may be determined using methods well known in the art and found in the foregoing references. Efficacy may be determined utilizing the same guidance in conjunction with the methods described below in the Examples. A pharmaceutically effective amount, therefore, is an amount that is deemed by the clinician to be toxicologically tolerable, yet efficacious. Efficacy, for example, can be measured by the induction or substantial induction of T lymphocyte cytotoxicity at the targeted tissue or a decrease in mass of the targeted tissue. Suitable dosages can be from about 1 mg/kg to 10 mg/kg.

The foregoing is not intended to have identified all of the aspects or embodiments of the invention nor in any way to limit the invention. The accompanying drawings, which are incorporated and constitute part of the specification, illustrate embodiments of the invention, and together with the description, serve to explain the principles of the invention.

All publications and patent applications mentioned in this specification are herein incorporated by reference to the same extent as if each independent publication or patent application is specifically indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

### LITERATURE CITED AND INCORPORATED BY REFERENCE

1. Ahima, R. S. et al., Role Of Leptin In The Neuroendocrine Response To Fasting, NATURE 382:250-52 (1996).
2. Arvat, E., et al., Preliminary Evidence That Ghrelin, The Natural GH Secretagogue (GHS)-Receptor Ligand, Strongly Stimulates GH Secretion In Humans, J. ENDOCRINOL. INVEST. 23, 493-495 (2000).
3. Baguisi A, (1999) et al., Production of Goats by Somatic Cell Nuclear Transfer, NATURE BIOTECH; 17: 456-461.
4. Barsh et la., From Agouti To POMC—100 Years Of Fat Blond Mice, NATURE MEDICINE 5 984-85, 1066-70. (1999).
5. Bouchard, C. et al., The Causes Of Obesity: Advances In Molecular Biology But Stagnation On The Genetic Front, DIABETOLOGIA 39, 1532-1533 (1996).
6. Butler A. A. et al., A Unique Metabolic Syndrome Causes Obesity In The Melanocortin-3 Receptor-Deficient Mouse, ENDOCRINOLOGY 141:3518-21 (2000).
7. Chambers, S. M. and Morris, B. J., Glucagon Receptor Gene Mutation In Essential Hypertension, NATURE GENETICS 12:122 (1996).
8. Chehab, F. F., et al., Early Onset Of Reproductive Function In Normal Female Mice Treated With Leptin, SCIENCE 275, 88-90 (1997).
9. Chen, G. et al., Disappearance Of Body Fat In Normal Rats Induced By Adenovirus-Mediated Leptin Genetherapy, PROC. NATL ACAD. SCI. USA 93, 14795-14799 (1996).
10. Cibelli J B, (1998) et al., Cloned Transgenic Calves Produced From Non-Quiescent Fetal Fibroblasts. SCIENCE; 280: 1256-1258.
11. Eipper B A, et al., Peptidylglycine alpha-Amidating Monooxygenase: A Multifunctional Protein with Catalytic, Processing and Routing Domains, (1993) PROTEIN SCIENCE 2, 489-497).
12. Halaas, J. L. et al. Weight-Reducing Effects Of The Plasma Protein Encoded By The Obese Gene, SCIENCE, 269: 543-46 (1995).
13. Horvath, T. L., et al., A. N. J. NEUROSCI. 19:1072-87 (1999).
14. Jain, M, et al., (2001). Targeted Inactivation of GαI Does Not Alter Cardiac Function or β-Adrenergic Sensitivity. AM. J. PHYSIOL. 280: 569H-575.
15. Kanehisa (1984) NUC. ACIDS RES. 12:203-213.
16. Kasinathan P, (2001) et al., Effect of Fibroblast Donor Cell Age and Cell Cycle on Development of Bovine Nuclear Transfer Embryos In Vitro, BIOL REPROD.; 64(5): 1487-1493.
17. Kasinathan P, (2001) et al., Production of Calves from G1 Fibroblasts, NATURE BIOTECH; 19: 1176-1178.
18. Kerr et al., NAT. BIOTECHNOL. 16:75-79, 1998.
19. Maffei, M. et al., Increased Expression In Adipocytes Of Ob RNA In Mice With Lesions Of Thehypothalamus And With Mutations At The Db Locus, PROC. NATL ACAD. SCI. USA 92, 6957-6960 (1995).
20. Maffei, M. et al., Leptin Levels In Human and Rodent, Measurement of Plasma Leptin and ob RNA In Obese And Weight-Reduced Subjects, NATURE MED. 1, 1155-1161 (1995).
21. Meng L, et al., (1997) Rhesus Monkeys Produced by Nuclear Transfer, BIOL REPROD. August; 57(2):454-9.
22. Mortensen, R, et al., (1992) Production of Homozygous Mutant ES Cells with a Single Targeting Construct. MOL. CELL. BIOL. 12, 2391-2395.
23. Nagy, A, et al., (1996) Targeted Mutagenesis: Analysis of Phenotype Without Germ-Line Transmission. J. CLIN. INVEST. 97: 1360-1365.
24. Ongeri E M, et al., (2001) Development of Goat Embryos After In vitro Fertilization and Parthenogenetic Activation by Different Methods, THERIOGENOLOGY June 1; 55(9):1933-45.
25. O'Rahilly et al., Insights Into Obesity And Insulin Resistance From The Study Of Extreme Human Phenotypes. EUROPEAN J. ENDOCRINOLOGY 147: 435-441 (2002).
26. Paris et al., BIOTECHNOL. APPL. BIOCHEM. 12:436-449 (1990).
27. Pedrazzini, T., et al., Cardiovascular Response, Feeding Behaviour And Locomotor Activity In Mice Lacking The NPY Y1 Receptor. NATURE MEDICINE 4:722-726 (1998).
28. Petersenn, S. et al., Genomic Structure And Transcriptional Regulation Of The Human Growth Hormone Secretagogue Receptor, ENDOCRINOLOGY 142 (6), 2649-2659 (2001).
29. Ravussin, E. et al., Relatively Low Plasma Leptin Concentrations Preceed Weight Gain In Pima Indians, NATURE MED. 3, 238-240 (1997).
30. REMINGTON'S PHARMACEUTICAL SCIENCES (16^{th} ed., Osol, A., editor., Mack, Easton Press. (1980)).
31. Sambrook et al., MOLECULAR CLONING—A LABORATORY MANUAL, Cold Spring Harbor Laboratory Press, (2nd Edition) 1989).
32. Shigeri, Y. et al., The Role of Central Neuropeptide, NEUROTRANSMISSIONS 15(126) (1998).
33. Shiiya, T., et al., Plasma Ghrelin Levels In Lean And Obese Humans And The Effect Of Glucose On Ghrelin Secretion, J. CLIN. ENDOCRNOL. METAB. 87:240-44 (2002).
34. Stephens, T. W. et al., The Role Of Neuropeptide Y In The Antiobesity Action Of The Obese Gene Product, NATURE 377:530-34 (1995).
35. Steppan & Lazar, Resistin & Insulin Resistance, TRENDS IN ENDOCRINOLOGY AND METABOLISM, 13/1: 18-21 (2002).
36. Tartaglia, L. A., et al. CELL 83:1263-71(1995).
37. Vaisse, C. et al. Leptin Activation Of Stat3 In The Hypothalamus Of Wild Type And Ob/Ob Mice But Not Db/Db Mice, NATURE GENET. 14:95-97 (1996).
38. Verploegen, S. A., et al. FEBS LETT. 405:237-40 (1997).
39. Volante M., et al., Ghrelin Immunoreactivity and Carcinoma Cell Proliferation Inhibition Effects in Fetal Thyroid and Follicular Tumors, AMERICAN JOURNAL OF PATHOLOGY, 2003; 162:645-654.
40. Wilding et al., Leptin & The Control Of Obesity, CURRENT OPINION IN PHARMACOLOGY 1:656-661 (2001).
41. Wilmut I, et al., (2002) Somatic Cell Nuclear Transfer, NATURE October 10; 419(6907):583-6.
42. Wilmut I, et al., (1997) Viable Offspring Derived From Fetal and Adult Mammalian Cells, NATURE Febuary 27; 385(6619):810-3.
43. Wren, A. M., et al., Ghrelin Enhances Appetite And Increases Food Intake In Humans, J. CLIN. ENDOCRINOL. METAB. 86, 5992-5995 (2001).
44. Yong Z and L Yuqiang, (1998) Nuclear-Cytoplasmic Interaction and Development of Goat Embryos Reconstructed by Nuclear Transplantation: Production of Goats by Serially Cloning Embryos, BIOL REPROD.; 58: 266-269.
45. Zou X, et al., (2002) Generation of Cloned Goats (Capra Hircus) From Transfected Foetal Fibroblast Cells, The Effect of Donor Cell Cycle, MOL REPROD DEV.; 61: 164-172.

### PATENTS CITED AND INCORPORATED BY REFERENCE

1. Meade et al., U.S. Pat. No. 5,750,172.
2. Meade et al., U.S. Pat. No. 4,873,316.
3. Stice et al., U.S. Pat. No. 5,945,577.
4. Meade, et al., U.S. Pat. No. 5,827,690.
5. DiTullio et al., U.S. Pat. No. 5,843,705.
6. Clark et al., U.S. Pat. No. 5,322,775.
7. Garner et al., U.S. Pat. No. 5,639,940.
8. Pedrazzini T et al., U.S. Pat. No. 5,817,912.
9. Van der Ploeg, L. H. T., et al., U.S. Pat. No. 6,639,123.
10. Cone R. D., et al., U.S. Pat. No. 6,476,187.
11. Wikberg, J., et al., U.S. Pat. No. 6,448,032.

1. A transgenically produced recombinant protein, encoded by a transgene DNA construct comprising a polypeptide domain which has a desired bioactivity comprising said transgenic protein or fragment thereof wherein said polypeptide domain retains its endogenous physiological activity and is useful in reducing the body-weight of a mammal.

2. The transgenic protein of claim 1, wherein said transgenic protein is the product of a contiguous coding sequence of DNA.

3. The transgenically produced protein of claim 1, wherein said protein is selected from the group comprising:
a) Leptin;
b) Glucagon;
c) Subpeptide PYY; and,
d) α-MSH.

4. A method of treating obesity or other disease condition medically related to obesity comprising administering to a mammal in need of such treatment a therapeutically effective amount of a compound of claim 1 or a prodrug thereof or a pharmaceutically acceptable salt of said compound or of said prodrug.

5. A method as recited in claim 4 wherein the amount of the Formula I compound is about 0.01 mg/kg/day to about 50 mg/kg/day.

6. A recombinant protein as recited in claim 1 wherein the mammal is a human.

7. A recombinant DNA vector comprising the nucleic acid sequence of the transgenic protein of claim 1.

8. A host cell transformed with said recombinant DNA vector of claim 7.

9. A recombinant DNA vector comprising the nucleic acid sequence of the recombinant transgenic protein of claim 1 wherein said vector is an expression vector comprising a promoter operably linked to a transgenic protein of claim 1.

10. The method of claim 1 wherein said DNA construct encoding a desired transgenic protein is actuated by at least one beta-casein promoter.

11. A recombinant transgenic protein produced by a method comprising:
(a) expressing the transgenic protein of claim 1 by a mammary epithelial cell; and
(b) recovering the protein.

12. The recombinant transgenic protein of claim 11, wherein said transgenic protein further comprises an N-terminal methionine.

13. The method of claim 4 wherein said disease condition is Diabetes Mellitus.

14. The method of claim 4 wherein said disease condition is Hypertension.

15. The method of claim 4 wherein said disease condition is Atheriosclerosis.

16. The method of claim 4 wherein said disease condition is Insulin Resistance.

17. The method of claim 13 wherein said disease condition is Diabetes Mellitus and treatment of said disease condition further comprises treatment with an effective amount of a second composition said second composition being selected from the group consisting of: insulin, insulin lente, and thiazolidinediones.

18. The recombinant protein of claim 1, wherein said transgenic protein is expressed by a prokaryotic cell.

19. The transgenic protein of claim 1, wherein said transgenic protein is expressed by a eukaryotic cell in in vitro cell culture conditions.

20. The transgenic protein of claim 19, wherein said transgenic protein is expressed by an animal cell.

21. The transgenic protein of claim 20, wherein said animal cell is a CHO cell.

22. The transgenic protein of claim 20, wherein said animal cell is a COS cell.

23. The transgenic protein of claim 19, wherein said transgenic protein is expressed by a yeast.

24. The transgenic protein of claim 23, wherein said yeast is Saccharomyces.

25. A method for the production of transgenic animals capable of producing a transgenic protein of interest comprising:
transfecting a non-human mammalian cell-line with a transgene DNA construct encoding a desired transgenic protein;
selecting a cell line(s) in which said transgene DNA construct has been inserted into the genome of that cell or cell-line; and
performing a first nuclear transfer procedure to generate a first transgenic animal heterzygous for the desired gene said animal being capable of expressing said transgenic protein of interest in its milk.

26. The method of claim 25 further comprising:
characterizing the genetic composition of said first heterzygous transgenic animal;
selecting cells homozygous for said desired transgene DNA construct through the use of a selective agent;
characterizing surviving cells using known molecular biolology methods; and
picking surviving cells or cell colonies cells for use in a second round of nuclear transfer or embryo transfer; and producing a second transgenic animal homozygous for said desired transgene DNA construct.

27. The method of claim 25 wherein said transgene DNA construct is operatively linked to a mammary tissue-specific promoter which enables the obesity related transgenic protein product of said transgene DNA construct to be produced in the milk of a transgenic non-human mammal.

28. The transgenic mammal of claim 27, wherein said promoter is the beta-casein promoter.

29. The resultant milk derived from the offspring of the methods of claim 28.

30. A method for treating a human disease or pathologic condition related to or caused by obesity with a composition of matter containing a recombinant transgenic protein, said transgenic protein encoding a human recombinant protein selected from the group consisting of: Leptin; Glucagon; subpeptide PYY; α-MSH; a dysfunctional Ghrelin Receptor; a dysfunctional MC-1 Receptor; a dysfunctional NPY Y1 Receptor; a dysfunctional NPY Y5 Receptor; a dysfunctional GAL1 Receptor; a dysfunctional GAL2 Receptor; a dysfunctional GAL3 Receptor; a dysfunctional Orexin Receptor 1; and a dysfunctional Orexin Receptor 2.

31. The method of claim 30 wherein said human disease is further comprised of the following disease conditions: diabetes mellitus; insulin resistance; leptin resistance; hypertension; atheriosclerosis; impaired glucose tolerance; and hyperphagia.

32. The method of claim 31 wherein said human disease condition is diabetes and said method of treatment includes administering a therapeutically effective amount of a recombinant protein of interest in addition to insulin via pump means.

33. A method for treating a human disease related to undesirably low body weight or weight loss with a composition of matter containing a recombinant transgenic protein, said transgenic protein encoding a human recombinant protein selected from the group consisting of: a dysfunctional Leptin receptor; Ghrelin; a dysfunctional glucagons Receptor; Agouti Signalling Protein; a dysfunctional MC-4 Receptor; NPY; Agouti Related Protein (AGRP); a dysfunctional MC-3 Receptor; Galanin; an Orexin A polypeptide; and an Orexin B polypeptide.

34. The method of claim 33 wherein said human disease is further comprised of the following disease conditions: anorexia nervosa; wasting disease; AIDS wasting; and Prader-Willi Syndrome.

35. A method for treating a human disease or pathologic condition related to or caused by obesity with two proteins, wherein a first protein is recombinant leptin and a second protein is a composition of matter containing a recombinant transgenic protein, said second protein is a human recombinant protein selected from the group consisting of: Glucagon; subpeptide PYY; α-MSH; a dysfunctional Ghrelin Receptor; a dysfunctional MC-1 Receptor; a dysfunctional NPY Y1 Receptor; a dysfunctional NPY Y5 Receptor; a dysfunctional GAL1 Receptor; a dysfunctional GAL2 Receptor; a dysfunctional GAL3 Receptor; a dysfunctional Orexin Receptor 1; and a dysfunctional Orexin Receptor 2.

36. The method of claim 35 wherein said human disease is further comprised of the following disease conditions: diabetes mellitus; insulin resistance; leptin resistance; hypertension; atheriosclerosis; impaired glucose tolerance; and hyperphagia.

37. The method of claim 36 wherein said human disease condition is diabetes and said method of treatment includes administering a therapeutically effective amount of recombinant proteins of interest in addition to insulin via pump means.

38. A method for treating a human disease related to undesirably low body weight or weight loss with two recombinant proteins, wherein a first protein is recombinant ghrelin and a second protein is a human recombinant protein selected from the group consisting of: a dysfunctional Leptin receptor; a dysfunctional glucagons Receptor; Agouti Signalling Protein; a dysfunctional MC-4 Receptor; NPY; Agouti Related Protein (AGRP); a dysfunctional MC-3 Receptor; Galanin; an Orexin A polypeptide; and an Orexin B polypeptide.

39. The method of claim 38 wherein said human disease is further comprised of the following disease conditions: anorexia nervosa; wasting disease; AIDS wasting; and Prader-Willi Syndrome.

40. A method as recited in claim 30 wherein the amount of the Formula I compound is about 0.01 mg/kg/day to about 50 mg/kg/day of each recombinant protein.

41. A method as recited in claim 33 wherein the amount of the Formula I compound is about 0.01 mg/kg/day to about 50 mg/kg/day of each recombinant protein.

42. A method as recited in claim 35 wherein the amount of the Formula I compound is about 0.01 mg/kg/day to about 50 mg/kg/day of each recombinant protein.

43. A method as recited in claim 38 wherein the amount of the Formula I compound is about 0.01 mg/kg/day to about 50 mg/kg/day of each recombinant protein.
